(19) United States
(12) Patent Application Publication
(10) Pub. No.: US 2018/0051317 A1
(43) Pub. Date: Feb. 22, 2018

(54) NON-RADIOACTIVE METHOD FOR DETERMINING THE CYTOLITIC ACTIVITY OF AN AGENT WITH RESPECT TO TARGET CELLS, USE THEREOF AND ASSOCIATED KIT

(71) Applicant: CLEAN CELLS, BOUFFERE (FR)

(72) Inventors: Véronique BONNAUDET, SAINT HILAIRE DE LOULAY (FR); Laurent BRETAUDEAU, MAISDON SUR SEVRE (FR); Alexis ROSSIGNOL, LES BROUZILS (FR)

(21) Appl. No.: 15/554,368

(22) PCT Filed: Mar. 1, 2016
(86) PCT No.: PCT/FR2016/050458

§ 371 Date: Aug. 29, 2017

(30) Foreign Application Priority Data
Mar. 5, 2015 (FR) 15 51835
Jul. 28, 2015 (FR) 15 57175

(51) Int. Cl. C12Q 1/66; G01N 33/50

(52) U.S. Cl. CPC C12Q 1/66; G01N 33/5014

(57) ABSTRACT

A non-radioactive method for the direct in vitro determination (control and quantification) of the cytolytic activity of an active agent with respect to target cells and/or a medium surrounding target cells, comprising the steps of genetically transforming target cells to express an enzyme exogenous to said target cells, exposing said genetically transformed target cells to the active agent and/or to said surrounding medium to be tested, which can result in the lysis of at least a portion of the target cells by releasing said exogenous enzyme into the extracellular medium, and measuring the activity of the exogenous enzyme released during the lysis of said target cells, characterised in that said exogenous enzyme is an enzyme having a molar mass less than or equal to 45 kDa (e.g. an Oplophorus gracilirostris luciferase), and of which the activity can be detected by luminescence or fluorescence. Application to the measurement of antibody-dependent cellular cytotoxicity (ADCC), complement-dependent cytotoxicity (CDC) and/or the measurement of apoptosis.

The present invention relates to the field of the measurement of cell death and more particularly to a method for the determination of the cytolytic action of active substances with respect to target cells and/or a medium surrounding said target cells.

In the industrial field, the regulations make it obligatory to characterize as completely as possible any phenomenon or product that may have consequences on the health of human or animal populations. In the vast context of the setting up of tests which measure cell death, called cytotoxicity tests, these phenomena or products can group together environmental phenomena, such as exposure to a source of irradiation or of pollution, chemical or biological products which are not for health purposes, or else products for therapeutic purposes.

It is in the field of biologically active substances, and more specifically in the context of substances for therapeutic purposes, that the characterization obligations are the most thorough. One of the main criteria to be characterized is that of the biological activity, also known as “potency”. Indeed, according to the regulatory definition of the European Medicines Agency (CHMP/BWP/157653/2007), the potency is the quantitative measurement of a biological activity based on an attribute of the pharmaceutical product associated with a relevant biological property of said product, this measurement having to reflect its biological activity in the clinical situation for which the product is designed. According to the regulations, a potency test must therefore be based on the direct measurement of the expected biological function of the product tested, coherent with its known or presumed mode of action. In other words, and in the case of a product, the purpose of which is to cause the death of a given cell population (tumor cell death in the case of an anti-cancer treatment, for example), a cytotoxicity test must be based on the direct measurement of the death of the target cell population. In the same way, the setting up of a test which searches for the absence (or the existence) of cytotoxic effects on a product not intended for therapeutic use must itself also be based on a method which specifically detects the death of target cells.

In the case of biological medicaments, in particular that of antibodies (or immunoglobulins, denoted Igs), for therapeutic use, cytotoxicity tests appear among the long list of quality control tests required by the regulations before these molecules are placed on the market.

According to the regulatory texts, these assays must reflect as much as possible the biological activity of the medicament in its clinical use, using its known or presumed mechanisms of action as a basis. However, the modes of action of antibodies are varied:
Mechanisms which are dependent on the Fab region of the antibody: direct action of antibodies via their antigen-binding activity. These mechanisms are specific to each antibody and can correspond to i) the neutralization of an antigen, ii) the neutralization of a membrane antigen by antagonism or iii) the agonistic action on a membrane antigen. In the latter two cases, the agonistic or antagonistic action on a membrane antigen can result in an inhibition of the growth of the target cell population and/or in the induction of its death by apoptosis and/or necrosis.
Fc region-mediated effector mechanisms: mechanisms common to all antibodies; their strength depends on the class and sub-class of the antibodies and also on their physicochemical structure (amino acid sequence, structure and composition of glycosylated chains, etc.). These Fc region-mediated effector functions will depend on the interaction of said Fc region with two major types of receptors specific for the immune system: i) the complement system via the interaction between the Fc region and the first component C1 of the proteolytic cascade of complement; ii) the antibody Fc region receptors for Igs (denoted FcR).

In many cases, in particular when the antibody is directed against a membrane molecule, the desired mechanism for observing the therapeutic effect is of lytic type (for example, in the case of a use in onco-hematology). The lysis can be obtained, for example, by induction of apoptosis/necrosis or of the inhibition of cell proliferation via agonism/antagonism phenomena, by activation of the complement system or by recruitment of cytotoxic cells expressing at the surface one of the FcRs (such as natural killer cells [NKs], macrophages, cytotoxic lymphocytes, polymorphonuclear cells, etc.). In such cases, the regulatory texts thus impose the setting up of a potency test which measures cell lysis in a manner that is biologically relevant and coherent with the mode of action envisioned in vivo.

A first objective of the invention is thus to provide a method for direct measurement of cell death (cytotoxicity) by measuring the amount released into the medium of an enzyme artificially introduced into said cells.

### PRIOR ART

Cytotoxicity, or cytolysis, tests are very widely used in biological, clinical and pharmaceutical research, for measuring cell death induced by any biologically active substance, by any biologically relevant mechanism, in order to screen a library of candidate molecules, to identify a mechanism of action or to characterize the biological activity of a pharmaceutical product for example (potency test).

A cytotoxicity test carried out in the context of a potency measurement consists in measuring the death of cells of interest (generally referred to as target cells) under experimental conditions which are coherent with the biological mechanism in question. In the context of therapeutic antibodies, three main mechanisms of action can be explored by means of this test: 1) induction of apoptosis by agonism or antagonism of a membrane molecule by the antibody; 2) activation of the complement system (CDC for “Complement-Dependent Cytotoxicity”) by the antibody adsorbed to the target cell; 3) a mechanism of lysis, by ADCC (for “Antibody-Dependent Cell-mediated Cytotoxicity”) or by phagocytosis, mediated by effector cells recruited via the interaction between the FcRs that they express at their surface and the Fc region of the antibodies adsorbed to the target cells. In order to carry out each of these tests, target cells are incubated with the antibody in question in the presence, respectively, 1) of the culture medium alone, 2) of the culture medium containing a source of complement or 3) of the culture medium containing appropriate effector cells.

There are at the current time several methods for direct measurement of cell death, that is to say of which the data measured come directly and effectively from the death of the target cells:

a). Method for release of a radioactive isotope (⁵¹Cr, ¹¹¹In, ³H). This method consists in incubating, prior to the cytolysis experiment in itself, the target cells in a solution of sodium chromate, which penetrates into the cells so as to bind into the intracellular proteins. When the cells die, their intracellular content is released into the supernatant. A measurement of the radioactivity of the supernatant thus allows a direct measurement of the amount of dead target cells, even if other types of cells (for example cytotoxic effector cells) have been mixed with the target cells during the assay, since only the target cells have been radio labeled. The background noise of the method (that is to say the strength of the signal in the absence of cell death) is minimal due to a very low spontaneous release of ⁵¹Cr over the average time that a cytotoxicity assay lasts (minimum of 3 to 4 hours). Furthermore, by virtue of its radioactive nature, the signal generated is very strong, which results in a high signal/noise ratio (of about 5 to 12). The method is thus very sensitive. Developed at the end of the 1960s, this method is still, at the current time, the reference method, owing to its specificity with respect to target cell death and to its high performance levels, in particular in terms of sensitivity. It also exhibits good capacities for high-throughput analysis owing to the stability of the signal over long periods of time (several hours to several days). On the other hand, it has the drawback of requiring a ⁵¹Cr-labeling time (incubation and washes), that cannot be shortened, of approximately one to two hours depending on protocols, significantly extending the duration of the experiment. Furthermore, the labeling adds variability to the assays, related to the amount and to the quality of the reagents added, and to the various incubation times and experimental steps. Finally, the regulatory restrictions associated with the use of radioactivity are increasingly burdensome and expensive, in terms of administrative authorization, of source management, of waste elimination, of labor law, of medical monitoring or of exposure of handlers. Moreover, several variants of this method have been described, using other radioisotopes, for example based on the use of tritium (³H) or indium (¹¹¹In) but involving the same radioprotection restrictions and thus subjected to the same limitations.

b). Calcein-acetoxymethyl (calcein-AM) method. This compound is permeable through lipid bi-layers (thus through the membranes of eukaryotic cells) by virtue of its acetoxymethyl radical and will thus penetrate into cells. Once inside, the acetoxymethyl radical is cleaved by intracellular enzymes (esterases), which then restore the fluorescence properties of the calcein. The calcein released into the supernatant serves to measure the amount of cells lysed. This method has advantageous capacities for high-throughput analysis while avoiding the problems of radioactivity. On the other hand, the spontaneous release of calcein is very high (approximately 40% of the maximum release) reflecting a high calcein-permeability of the plasma membrane, which results in a high background noise of the method. Since the fluorescent signal emitted by calcein is weak, the signal/noise ratio is not very favorable, at around 2.5. This method thus has a sensitivity and performance levels that are not very high and comprises a step of labeling target cells with calcein-AM which increases the overall experimental time of the assay from one hour to two hours and further amplifies its variability.

c). Method based on the use of lanthanides, such as europium (Eu³⁺) or terbium (Tb³⁺), complexed with a chelate or amplifier of fluorescence, such as diethylenetriaminopentaacetate (DTPA) or 2,2′:6′,2″-terpyridine-6,6″-dicarboxylic acid (TDA), similar in their principle to the ⁵¹Cr and calcein-AM methods. For example, in the DELFIA technology (sold by Perkin Elmer, Boston, Mass.), target cells are loaded with a fluorescence-amplifying ligand, BATDA (bis[acetoxymethyl] 2,2′:6′,2″-terpyridine-6,6″-dicarboxylate), which penetrates through plasma membranes. In the cell, the ester bonds are hydrolyzed by esterases to form a hydrophilic ligand, TDA, which is in theory not very membrane-permeable (after addition of probenecid, an inhibitor of MDR, “Multi Drug Resistance” transporter) and released into the extracellular medium during cytolysis. After removal of the supernatant, a europium (Eu) solution is added so that said europium complexes with the free TDA so as to form a fluorescent chelate (EuTDA). The measurement of this signal, of TRF (Time-Resolved Fluorescence) type, is indicative of the amount of lysed cells. In another case, the target cells are labeled with europium and its release into the supernatant is measured by the addition of the DTPA chelate. These methods are non-radioactive and exhibit a capacity for high-throughput analysis. On the other hand, the spontaneous release of free TDA or free europium is quite high and variable depending on the cell type: the maximum release for Eu³⁺ is double that with ⁵¹Cr. In certain cases (FIG. 1), the strength of the spontaneous release is virtually equal to that of the specific signal and exhibits a variability in the strength of the signal associated with the cell type in question or with the physiological state of the cell (which conditions for example the nature and the activity of its esterases). This method itself also requires an experimental time for labeling the target cells with the europium or the BATDA, and also a time for incubation of the supernatants with the second component (respectively TDPA or Eu³⁺) which increase the overall experimental time of the assay and its variability through the addition of supplementary steps. All of these limitations generate quite a high variability of the Eu³⁺ assay, and also make it impossible to use it with a certain number of cell types, which makes its use and its validation difficult in an industrial context.

d). Methods using the flow cytometry technique (and its derivative of flow cytometry imaging) for measuring the frequency of live and/or dead cells during the assay. These methods have in common the use of a combination of one or more fluorescent markers for distinguishing the target and effector cell populations. Alternatively, some authors have used definitive labeling of the target cells by genetically transforming them to stably express a fluorescent protein. This labeling of the target populations is coupled to detection of the cell viability by using appropriate labels (generally DNA-intercalating agents which are impermeable with respect to the membranes of live cells but which penetrate into apoptotic or dead cells). The combination of these labels makes it possible to determine a frequency and/or a number of dead or live cells within a given population (generally the target cells). Although these methods are specific for the cell death mechanism, they do not enable a high-throughput analysis because of the technical restrictions associated with flow cytometry. For example, some probes can induce cross-labeling during the reaction (by exchange between the various cell types of probes bonded by non-covalent bonds). Depending on the methods and equipment, the acquisition time of a flow cytometry sample can take several seconds to several tens of seconds. The non-simultaneous acquisition of the samples which results therefrom means that several tens of minutes can elapse between the first and the final sample of a series, during which time the physiological conditions of the cells can change. These various elements have significant consequences on the variability of the assays and, in order to achieve with these methods performance levels that are compatible with the standards of the pharmaceutical industry, the analysis cannot exceed a few samples (in practice no more than three) tested simultaneously, which corresponds to a low throughput.

e). Method based on counting under a microscope by an operator or by an automated system. The distinction between dead and live cells and the counting thereof are carried out through the use of a vital die, for example trypan blue or eosin. Nevertheless, these microscopic methods do not make it possible to discriminate between several distinct cell types that would have been mixed with the needs of the assay (in the case of an ADCC measurement for example), unless fluorescence microscopy methods are used. This then amounts to methods similar to those described for flow cytometry, but which use laborious counting methods and the already mentioned drawbacks of which are further amplified. These methods, which are slow, not very reproducible and not at all suitable for high-throughput analysis, are not in practice used in the process of potency assays.

f). Impedance variation: a certain number of electronic methods exist for measuring the detachment of cells that are adherent to a support (this phenomenon possibly being due to the death of the cells, non-exhaustively). These methods are generally based on the measurement of an impedance variation induced by the amount and/or the physiological state of the cells present on a suitable support. Nevertheless, such methods are not suitable when several different cell types are mixed and when the death of a single type must be measured (in the case of ADCC assays for example). Furthermore, these methods require the use of adherent target cells, and expensive equipment, often associated with a complex experimental device. In addition especially, the measurement of cell detachment does not correspond to the mechanisms of cytolysis; it can, at best, be correlated therewith. Thus, their use in an industrial context is therefore difficult.

g). Methods based on the detection of components naturally expressed in the cytoplasm of eukaryotic cells. Such molecules must be free in the cytoplasm (that is to say not included in vesicles nor bound to organelles) in order to be able to released into the supernatant in the event of lysis of the cell, but they must not be secreted into the extracellular medium when the cells are in their physiological state. Their presence in the supernatant must be easily measurable by common methods and they must be strongly expressed (i.e. a large number of molecules per cell), this being by as many different cell types as possible (ubiquitous molecules). There are a very large number of described solutions or of commercially available kits claiming measurement of cytotoxicity by such methods. The visualizing technologies are generally based on the addition to the supernatant of one or more buffers, substrates, enzymes and/or reagents for carrying out the quantitative measurement by generating a final molecule that can be measured by bioluminescence, chemiluminescence, colorimetry or fluorescence. It should be noted that, in most cases, these tests can also be used “the other way round” to determine the overall viability of the cell population. These methods have the advantage of not requiring specific labeling of the target cells (decreasing the risk of variability and also the handling time) and, by virtue of their nature, allow high-throughput analyses. Nevertheless, the major and unacceptable drawback of all these tests based on the release of a ubiquitous molecule for measuring cell lysis is that they are not longer relevant once several cell types are mixed for carrying out the assays. This is because it is then impossible to distinguish which of these cell types has participated in, and in what proportion, the release of the molecule. This is, for example, the case with ADCC assays in which target cells and effector cells must be mixed in order to observe the cytotoxic effect. The effector cells which are dead during the assay, in relation to their usual mortality rate or via mechanisms associated with the assay in itself (such as exhaustion or redirected lysis for example), will, themselves also, contribute to the signal. In this sense, they are not methods specific to target cell lysis. In addition, some molecules to be measured are not sufficiently stable during the period of the assay (for example ATP has a very short half-life in the extracellular medium), others exhibit a high background noise and/or a weak signal strength, resulting in a lack of sensitivity of the method.

In the light of these elements, another objective of the invention is thus to provide a non-radioactive cytotoxicity method, in order to dispense with the numerous regulatory and health constraints.

Another object of the invention is to provide a cytotoxicity method which does not require extemporaneous labeling of the target cell (so as not to involve a loss of experimental time and an increase in overall variability of the assay by adding several variable steps), while at the same time preserving a direct and specific measurement of the lysis of said cell.

In order to overcome the abovementioned drawbacks and to bypass the need for extemporaneously labeling the target cell, while at the same time preserving a direct and specific measurement of the lysis of said cell, Schafer et al. described in 1997 (Journal of Immunological Methods, 204 pp 89-98, 1997) a genetic transformation method for obtaining target cell lines stably expressing an intracytoplasmic exogenous enzyme. In this method, the death of said cells in theory had to allow the release of this enzyme into the extracellular medium, the amount of enzyme then being measured by an appropriate method. These authors first of all carried out the transformation of a target line with the F-Luc (Firefly Luciferase) gene under the control of the beta-actin promoter. However, the lifetime of F-Luc in the extracellular medium is very short (half-life of 30 minutes) and is incompatible with an ADCC or CDC assay, the mechanism of action of which requires 2 to 4 hours to reach the maximum of lysed cells. They then evaluated the transformation of the target cells with the beta-galactosidase gene and compared these performance levels to that of the conventional radioactive chromium-labeling method. Although beta-galactosidase proves to be more stable than F-Luc, and that the signal/noise ratio is better with beta-galactosidase than with the radioactive chromium method, the method with beta-galactosidase underestimates by approximately 30 to 40% the amount of dead cells, probably because of an incomplete release of this enzyme during the death of the cells. It is not therefore applicable for effectively measuring the death of target cells in a non-radioactive test for characterizing therapeutic antibodies intended to meet the regulatory requirements, for example in the context of the characterization of ADCC and CDC activities.

More recently, other authors have described the use of target cells constitutively expressing F-Luc in an ADCC test, based on a different principle (Alpert et al., J. Virol. 86:12039, 2012; Fu et al., PLoS ONE 5:e11867, 2010): quantification of the luminescent signal serving to evaluate the amount of live cells. This is not therefore a method of direct measurement of target cell death, which is furthermore subject to certain limitations. Thus, since the half-life of F-Luc is short, and since the assay is relatively long (8 hours), the F-Luc activity is sensitive to variations such as any disturbance of the physiological state of the cells during the assay (decrease in synthesis, increase in catabolism), without this variation being directly linked to a modification of cell mortality and with it in fact generating a first level of variability of the method. Likewise, since the measurement is carried out homogeneously on the cell suspension lysed after the assay, it is highly likely that the F-Luc produced during the assay (or at the very least during the final hours, given its short half-life) and released by the dead cells into the supernatant will generate a second level of variability by adding its signal to that of the purely intracellular F-Luc. The presence of a residual F-Luc activity in the supernatant was moreover revealed by the authors. Finally, this method requires very long experimental times (at least 8 hours) for the loss of signal to be sufficiently significant. This method is not therefore applicable to the functional characterization of therapeutic antibodies in the context of “potency” assays in accordance with the regulatory requirements.

Another objective of the invention is thus to overcome the abovementioned drawbacks and to provide a method which includes a genetic transformation step in order to obtain target cell lines stably expressing, throughout the assay, an intracytoplasmic exogenous enzyme, the death of said cells allowing the (virtually total) release of this enzyme into the extracellular medium.

A further object of the invention is to provide a method of direct measurement which is sensitive and specific for target cell death (and not a measurement of the disappearance of the live cells), which is in particular applicable to the functional characterization of therapeutic antibodies in the context of “potency” assays.

These objectives, and also others, are achieved by means of the non-radioactive method according to the present invention for the direct in vitro determination (control and quantification) of the cytolytic action of an active agent with respect to target cells and/or a medium surrounding target cells, comprising the successive steps below:
i) Genetically transforming target cells to express an enzyme exogenous to said target cells,
ii) Exposing said genetically transformed target cells to the active agent and/or to said surrounding medium to be tested, which can result in the lysis of at least one portion of the target cells while releasing said exogenous enzyme into the extracellular medium,
iii) Measuring the activity of the exogenous enzyme released during the lysis of said target cells,

characterized in that said exogenous enzyme is an enzyme which has a molar mass of less than or equal to 45 kDa, and the activity of which can be detected by luminescence or fluorescence.

Thus, the release of this exogenous enzyme, in particular because of its reduced size, is representative of the lysis of the target cells and gives results, in terms of background noise, of maximum lysis, of specific lysis and of overall performance levels, which are similar to or better than the ⁵¹Cr reference method, as will be demonstrated below in the examples.

Advantageously, the molar mass of the exogenous enzyme is less than or equal to 30 kDa, preferably less than or equal to 25 kDa, more preferably less than or equal to 20 kDa.

Advantageously, said exogenous enzyme has an enzymatic activity that is stable in the extracellular medium of steps ii) and iii) of said method for at least 3 hours, preferably for at least 24 hours and more preferably for at least 48 hours, at temperatures of between 34° C. and 40° C.

According to one preferred embodiment, said exogenous enzyme is a luciferase.

More particularly, said exogenous enzyme has a peptide sequence which can exhibit at least 60% homology, preferably at least 80% homology, with the wild-type peptide sequence of the 19 kDa subunit of the luciferase produced by the shrimp Oplophorus gracilirostris.

Preferably, said exogenous enzyme is in a form with optimized activity and stability, sold by the company Promega under the name NanoLuc®, referred to throughout the text as nanoluciferase.

Such an enzyme is in particular stable in the extracellular medium over periods of time compatible with the types of assay envisioned, that is to say at least 24 hours.

The amount of exogenous enzyme released is measured by cleavage of a substrate specific for said enzyme, the substrate preferably being of the coelenterazine family, more particularly of the family of furimazine and derivatives thereof.

The medium to be tested that surrounds the target cells can comprise a biological agent and/or a chemical agent and/or a physical agent which is (are) potentially active with respect to said target cells.

The method thus comprises, as first step i), genetically transforming a relevant line of target cells in order to make it stably express said exogenous enzyme, segregated in the cytoplasm under physiological conditions.

According to a first embodiment of the invention, the biological agent is an antibody, preferably chosen from monoclonal antibodies, in particular for therapeutic purposes.

In the case of antibodies or related molecules, the choice of the support target cell is made according to the target of the antibody to be tested. With each new experimental model (for a given antigenic target), a new target cell is developed, unless one and the same target naturally or artificially expresses several relevant antigens.

Several options are possible for the choice of these target cells.

The first option is to select, among the cell lines available from the various biological material deposit banks, a relevant line for the model to be developed. Some of these lines will be given, by way of example, in the next paragraph, limited to a few antigenic targets for which commercially available antibodies currently exist. It is clearly understood that other lines are possible, for the antigenic molecules mentioned but also for other antigenic targets not mentioned but for which antibodies are to be developed or are undergoing development.

In the case of antibodies directed against molecules expressed mainly by normal or pathological B lymphocytes, such as CD19, CD20 or IL-6R (CD126), all the lines of B lymphocyte or lymphoma type are possible candidates, for example the WIL2, WIL2-S, Daudi, Raji, Ramos, JY, MC116, GA-10, DOHH, ARH-77, SU-DHL and Z138 lines or derivatives thereof. In the case of antibodies directed against molecules expressed mainly by normal or pathological T lymphocytes, such as CD3, CD25 or LPAM (Lymphocyte Peyer's Patch Adhesion Molecule or alpha[4]beta[7]-integrin), all the lines of T lymphocyte or lymphoma type are potential candidates, for instance the Jurkat, DERL, HD-MAR-2, HH, SR-786, SUP-T1, Loucy, CCRF-CEM and HUT-78 lines or derivatives thereof. The antibodies may also be antibodies directed against molecules expressed mainly by normal or pathological cells of lymphoid origin, such as human CTLA-4 (CD152), PD-1 (CD279) or CD30; in this case, all the B and T lymphocyte lines would be potential candidates, thus for example all the B and T lines already previously mentioned. The antibodies may also be antibodies directed against molecules expressed mainly by normal or pathological cells of lymphoid and myeloid origin, such as human CD52, VLA4 (CD49d) or LFA-1 (CD11a); in this case, any cell line of lymphoid or myeloid linage would be suitable, that is to say, in addition to the B and T lymphocyte lines already previously mentioned, myeloid lines such as THP-1, HL-60 or U-937 or derivatives thereof, for example. The antibodies may also be antibodies directed against molecules expressed mainly by carcinoid cells, such as anti-EGFR (for Epidermal Growth Factor Receptor, also denoted HER1 or ErbB1), EGFR2 (or HER2, or ErbB2), EGFR-3 (or HER3, or ErbB3) or EpCAM (CD326): any line derived from carcinoid tumors is therefore a potential candidate, for example HCC1954, SKBR3, SKOV3, Caco-2, HeLa, MCF-7, PC-3 or derivatives thereof. The antibody may also be an antibody directed against the TNF-alpha molecule: the T lymphocyte lines or lines of myeloid type as previously described are therefore potential candidates. The antibody may also be an antibody directed against the VEGF (Vascular Endothelial Growth Factor) molecule or its VEGFR receptor: lines described for expressing these molecules would then be suitable, for example the A375, M21, Hoc-7, Panc-3, D283Med, DAOY and D341Med lines, or derivatives thereof.

The other possible option for the construction of relevant target cell lines is to have one and the same line of cells expressing the chosen exogenous enzyme, into which one or more antigenic targets of interest have just been integrated, in a second step, by conventional genetic transformation techniques. In this case, any cell line may be suitable provided that it has a non-zero genetic transformation potential. Preferably, the chosen line will not express potential human target antigens that could disrupt the expression of the molecules introduced by genetic engineering, and therefore increase the variability of expression of the molecules, and therefore increase the general variability of the assay. For example, these cells may be of non-human origin, such as the CHO, Sp2/0, NSO or NIH3T3 cells or derivatives thereof, or of human embryonic origin, such as the HEK293, IMR-90 or NTera2 cells or derivatives thereof.

According to a second embodiment of the invention, the chemical and/or physical agent is chosen from chemotherapy agents or anti-cancer molecules, preferably cytotoxic molecules or molecules of the protein kinase inhibitor family.

Advantageously, the method has consisted in causing the target cells to express nanoluciferase.

This nanoluciferase is a protein derived from the 19 kDa subunit of a luciferase extracted from the deep-sea shrimp Oplophorus gracilirostris, the second subunit being a 35 kDa protein (proteins described in the article by S. Inouye et al., Secretional luciferase of the luminous shrimp Oplophorus gracilirostris: cDNA cloning of a novel imidazopyrazinone luciferase, in FEBS Letters, 481 (2000) 19-25). This nanoluciferase has been optimized and sold by the company Promega under the name NanoLuc® (and described in M. Hall et al. Engineered luciferase reporter from a deep sea shrimp utilizing a novel imidazopyrazinone substrate in ACS Chemical Biology 2012, 7, 1848-1857) for the purpose of improving the reporter gene systems for i) systems for studying protein-protein or protein-ligand interactions; ii) systems for studying protein stability; iii) systems of biosensors which donate energy to a third acceptor molecule (BRET); iv) in vivo imaging systems; v) systems for studying viral load and replication; and vi) reporter assay systems for cell signaling (studies of gene expression or of monitoring of intracellular protein metabolism). The following have in particular been described to date: intracellular use as a reporter gene (signaling pathways, receptor binding, for example), imaging on live cells/animals (labeling of cells to monitor their biological progression) or the labeling of bacteria or of viruses for monitoring infections; the coupling of nanoluciferase for a protein of interest for measuring its internalization or its secretion (Norisada et al., Biochem. Biophys. Res. Comm. 449:483, 2014). These systems have in common the fact that they couple the coding sequence of nanoluciferase to that of another protein of interest and/or to the sequence of a genetic promoter that is specifically activated by the signaling pathway studied, and/or the fact that they are restricted to intracellular detection of the molecule.

Conversely, the approach of the inventors has consisted in providing a method in which the target cells are genetically transformed in order to transiently or constitutively express said exogenous enzyme (for example nanoluciferase) in a cytoplasmic and non-secreted form, more particularly in causing said enzyme to be expressed alone and in freeform in the cytoplasm of the target cells and in measuring only and specifically the amount of molecules released into the extracellular medium following the lysis of the cells by mechanisms such as ADCC, CDC, apoptosis or detergent lysis.

To this effect, the step of genetically transforming the target cells advantageously comprises introducing into said cells an expression vector carrying the coding sequence of the exogenous enzyme and a promoter of constitutive type which allows its transcription in a cell, such as a eukaryotic cell.

Preferably, the vector may be a viral vector or a plasmid vector, preferably a plasmid vector.

Advantageously, the exogenous enzyme expression vector comprises an antibiotic resistance gene. This antibiotic resistance gene allows selection of the eukaryotic target cells having integrated the vector (cells known as “transformed”). The selectable antibiotic is in particular capable of eliminating the eukaryotic cells not carrying the resistance gene. This antibiotic resistance gene may be, in particular, a gene for resistance to geneticin (G418), puromycin, blasticidin, hygromycin B, mycophenolic acid or zeocin, preferably a puromycin resistance gene.

Preferably, the introduction of said expression vector into said cells is carried out by infection with viral particles carrying the exogenous enzyme gene when the expression vector is a viral vector, or by chemical or physical methods when the expression vector is a plasmid vector. When the vector is a plasmid vector, the introduction into the target cells is advantageously carried out by electroporation.

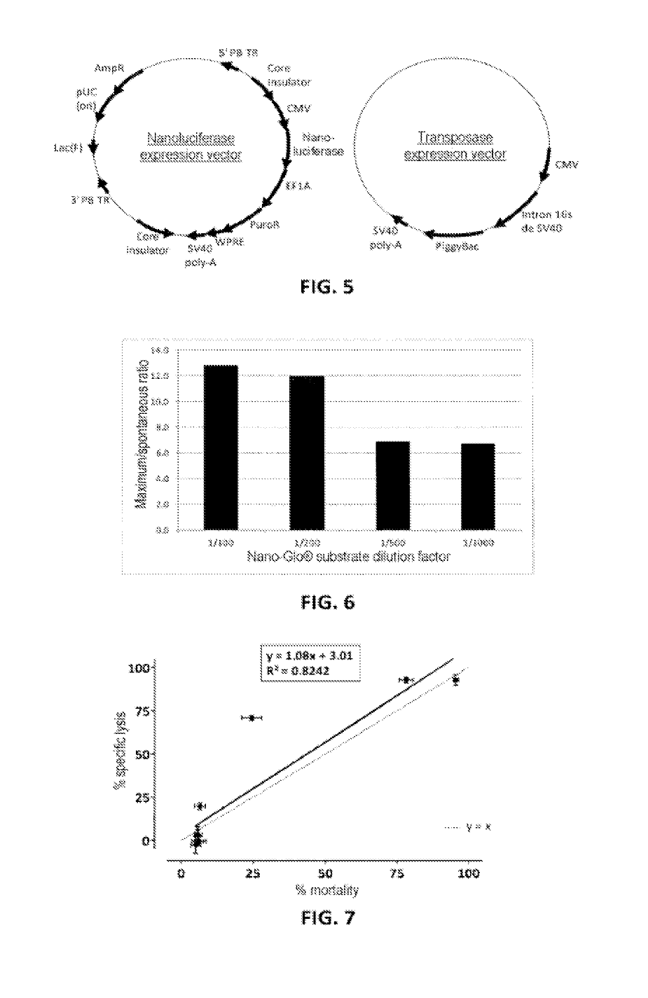

These various constitutive elements are assembled according to the conventional molecular biology cloning methods: use of restriction enzymes optionally coupled to polymerase chain reactions (PCRs) in order to isolate the DNA sequences of interest, and religation of the various constituents by means of DNA repair or synthesis enzymes (ligases or DNA polymerases, for example). A system is then provided which allows the genetic transformation of a target cell so that it expresses the exogenous enzyme (for example nanoluciferase) in its cytoplasmic compartment. An example of a cell transformation system based on the piggyBac transposase is given in FIG. 5.

Although this is not essential to the implementation of the present invention, it is recommended to subsequently carry out a step of cloning these cell mixtures. This makes it possible to obtain a line of target cells expressing nanoluciferase and derived from a single cell (or clone), which will promote the uniformity and the reproducibility of the results of the cytotoxicity test.

The method according to the invention has an advantageous use for measuring antibody-dependent cell-mediated cytotoxicity (ADCC), measuring complement-dependent cytotoxicity (CDC) and/or measuring apoptosis.

The present invention also relates to a kit for carrying out the method described above, or the use thereof, comprising:
a line of target cells expressing the exogenous enzyme,
cytotoxic effector cells for carrying out an ADCC test and/or a source of complement for carrying out a CDC assay,
a substrate which activates said exogenous enzyme so as to produce an emission of light, and any associated buffer solutions,
an instruction sheet.

The present invention will now be described in greater detail by means of the non-limiting examples below, mentioned by way of illustration, with reference to the figures in which:

FIG. 1 shows results obtained with the calcein-AM method (DELFIA) of the prior art (comparative example A);

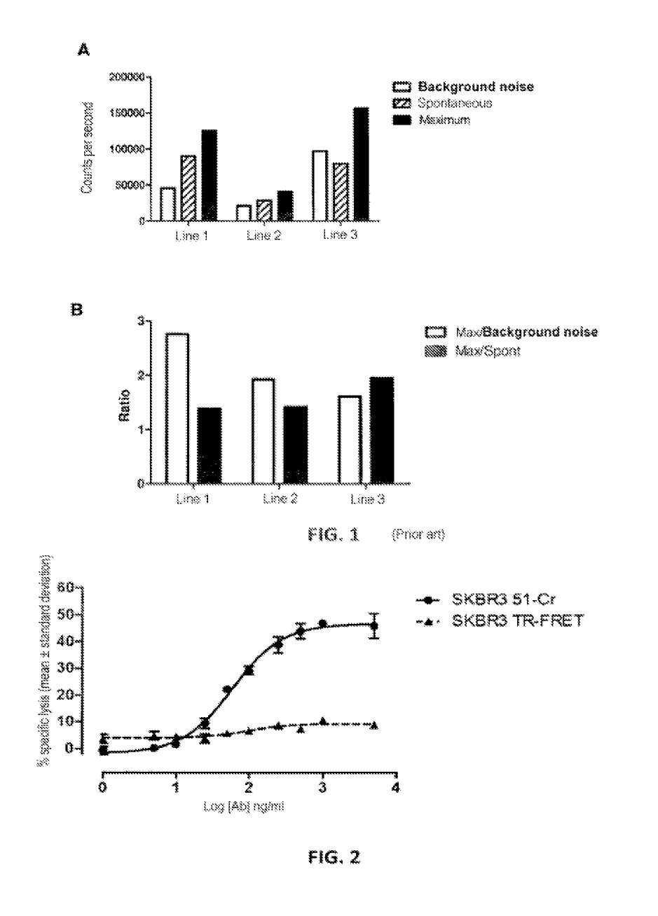

FIG. 2 shows the comparison between the ⁵¹Cr method and a TR-FRET method using two antibodies, one being a donor antibody and one being an acceptor antibody (comparative example B);

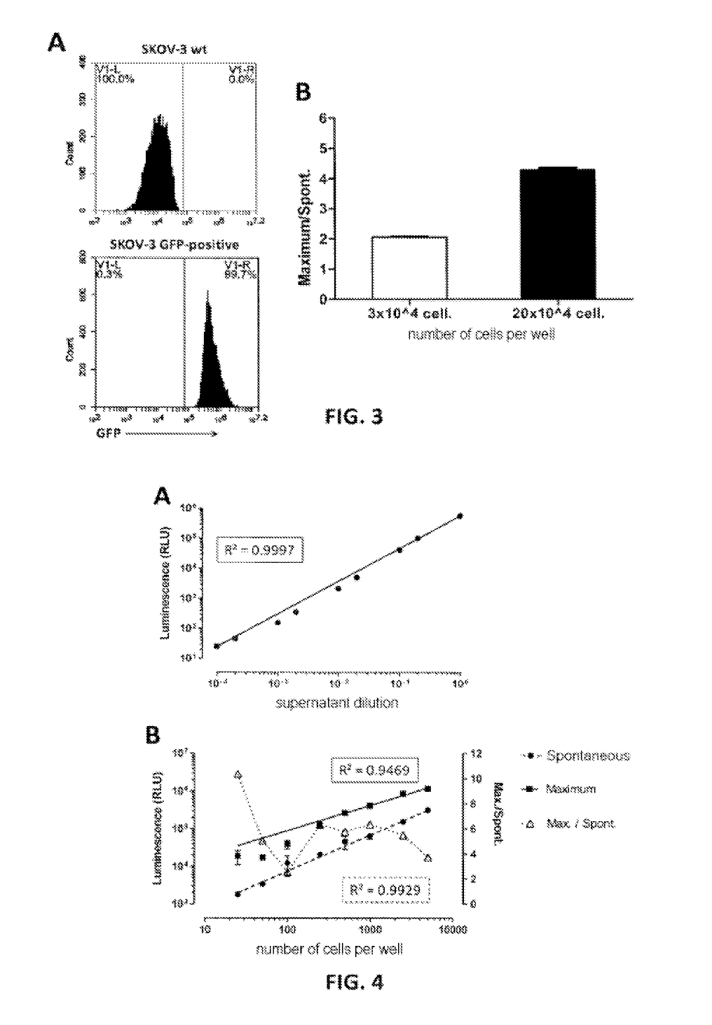

FIG. 3 shows the preliminary results from a TR-FRET method using a donor antibody and GFP (comparative example C);

FIG. 4 shows the linearity of the luminescent signal generated by the nanoluciferase and also the linearity of the relationship between the number of cells in the wells after lysis with triton X-100 and the strength of the signal;

FIG. 5 shows the map of the plasmids used to transform the target cells;

FIG. 6 illustrates the strength of the luminescent signal obtained as a function of the dilution factor applied to the nanoluciferase substrate;

FIG. 7 presents the correlation between the percentage mortality measured by flow cytometry and the percentage specific lysis obtained by the nanoluciferase method;

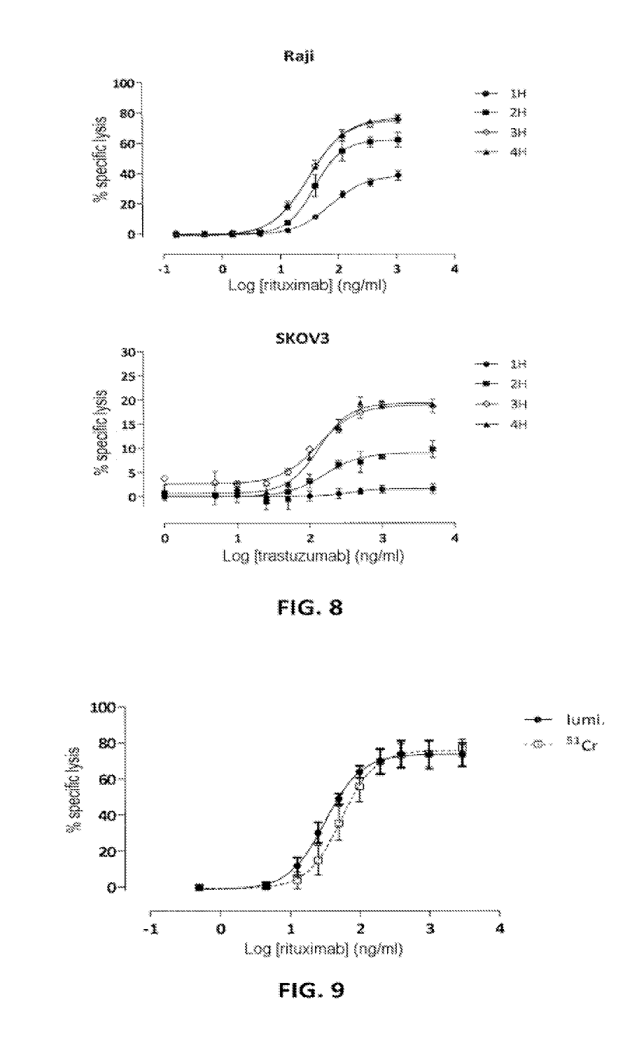

FIG. 8 describes the influence of the reaction time on the measurement of the target cell-specific lysis in an ADCC assay implementing the method;

FIG. 9 shows the direct comparison of the nanoluciferase (“lumi.”) and chromium-51 (“51Cr”) methods on the Raji model in an ADCC assay (N=3 experiments, carried out by 2 different operators: 2 experiments by operator No. 1 and 1 experiment by operator No. 2);

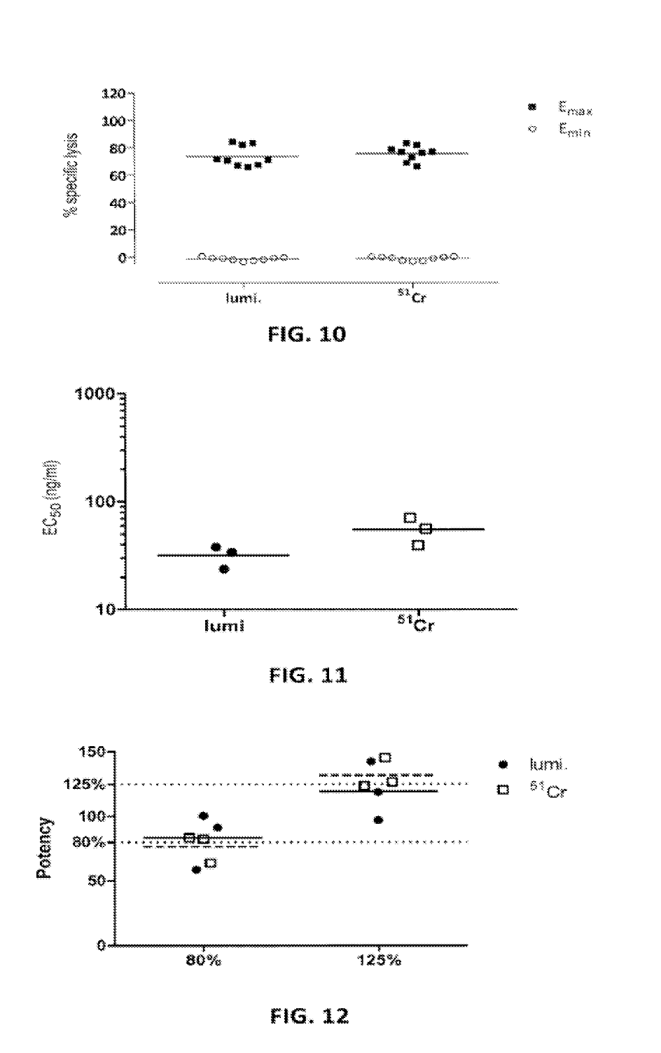

FIG. 10 shows the dispersion of the Emax and Emin data of the ADCC experiments described in FIG. 9;

FIG. 11 shows the dispersion of the EC50 data of the three ADCC experiments described in FIG. 9;

FIG. 12 shows the results of the calculation of potency for the 3 ADCC experiments described in FIG. 9;

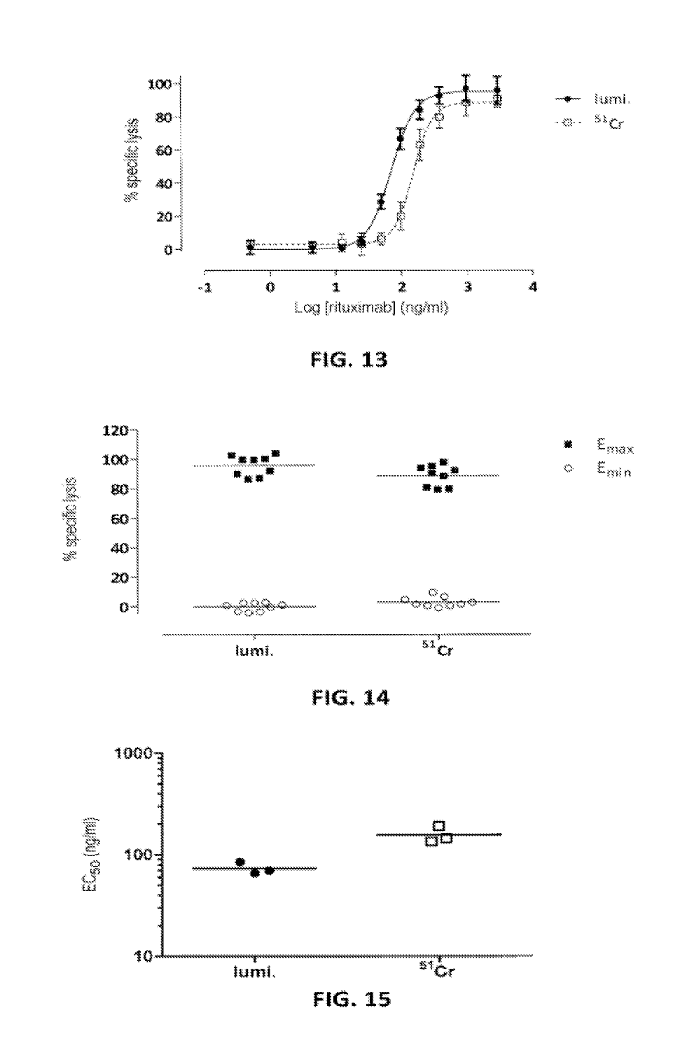

FIG. 13 shows the direct comparison of the nanoluciferase (“lumi.”) and chromium-51 (“51Cr”) methods on the Raji model in a CDC assay (N=3 experiments, carried out by 2 different operators: 2 experiments by operator No. 1 and 1 experiment by operator No. 2);

FIG. 14 shows the dispersion of the Emax and Emin data of the CDC experiments described in FIG. 13;

FIG. 15 shows the dispersion of the EC50 data of the three CDC experiments described in FIG. 13;

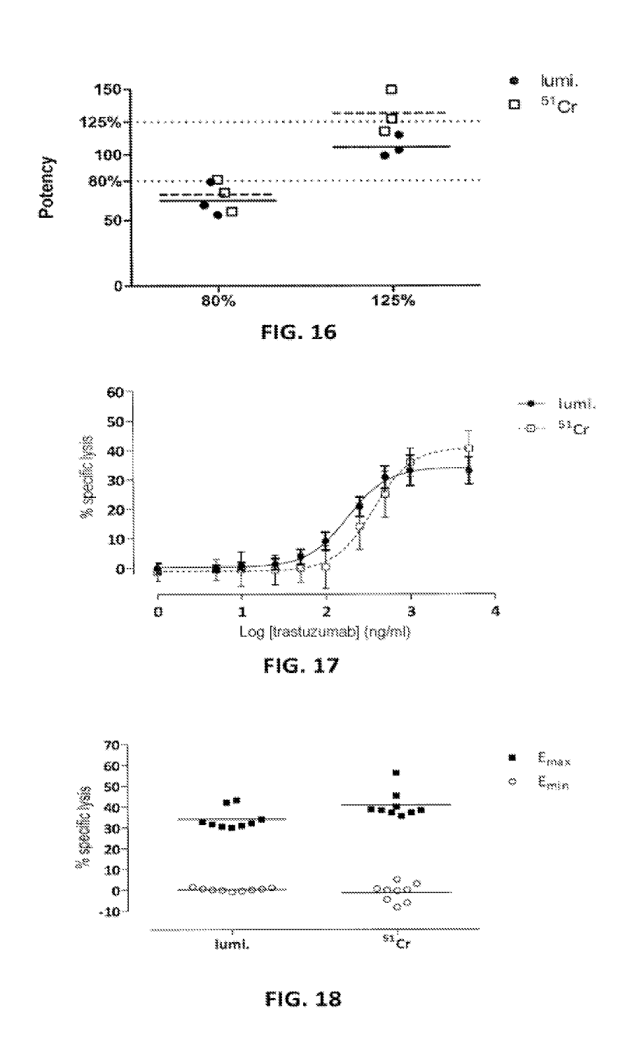

FIG. 16 shows the results of the calculation of potency for the 3 CDC experiments described in FIG. 13;

FIG. 17 shows the direct comparison of the nanoluciferase (“lumi.”) and chromium-51 (“51Cr”) methods on the SKOV3 model in an ADCC assay (N=3 experiments, carried out by 2 different operators: 2 experiments by operator No. 1 and 1 experiment by operator No. 2);

FIG. 18 shows the dispersion of the Emax and Emin data of the ADCC experiments described in FIG. 17;

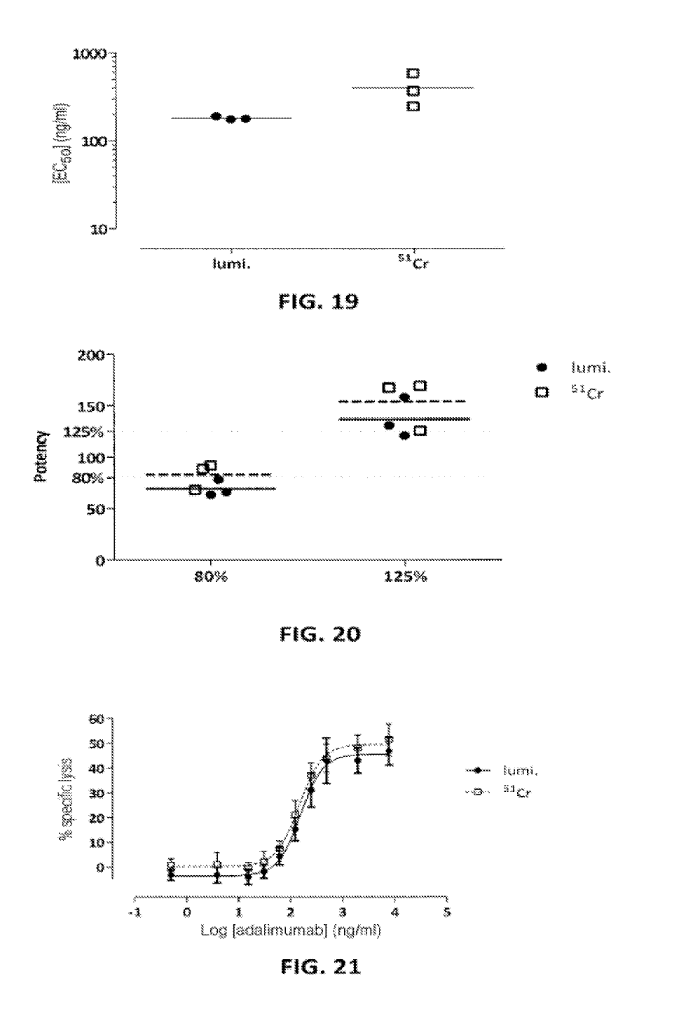

FIG. 19 shows the dispersion of the EC50 data of the three ADCC experiments described in FIG. 17;

FIG. 20 shows the results of the calculation of potency for the three ADCC experiments described in FIG. 17;

FIG. 21 shows the direct comparison of the nanoluciferase and chromium-51 methods on the CHO-TNF-alpha model in a CDC assay;

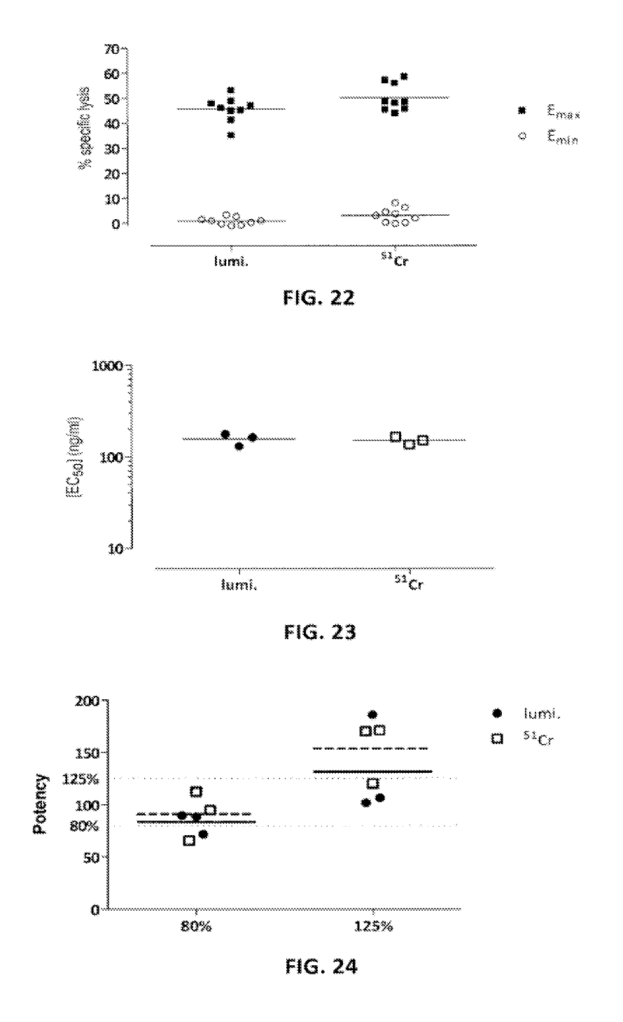

FIG. 22 shows the dispersion of the Emax and Emin data of the CDC experiments described in FIG. 21;

FIG. 23 shows the dispersion of the EC50 data of the three CDC experiments described in FIG. 21;

FIG. 24 shows the results of the calculation of potency for the 3 CDC experiments described in FIG. 21;

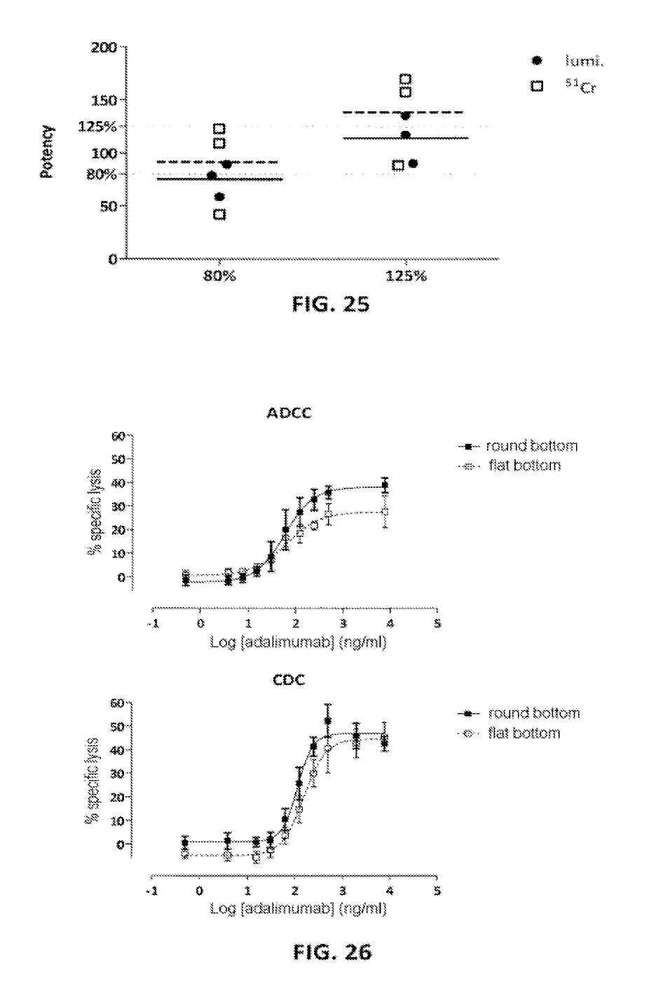

FIG. 25 shows the results of the calculation of potency for the 3 ADCC experiments carried out in the CHO-TNF-alpha model;

FIG. 26 summarizes the experiments for evaluating the influence of the cell interactions on the measurement of the target cell-specific lysis in an ADCC or CDC assay implementing the nanoluciferase method;

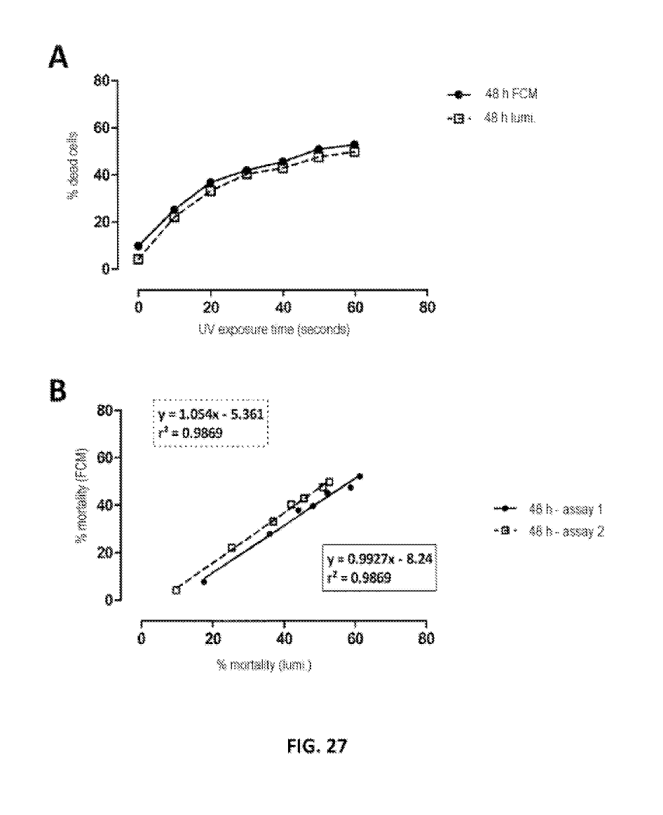

FIG. 27 illustrates the capacity of the nanoluciferase release method to measure the target-cell death induced by a pro-apoptotic treatment of physical type;

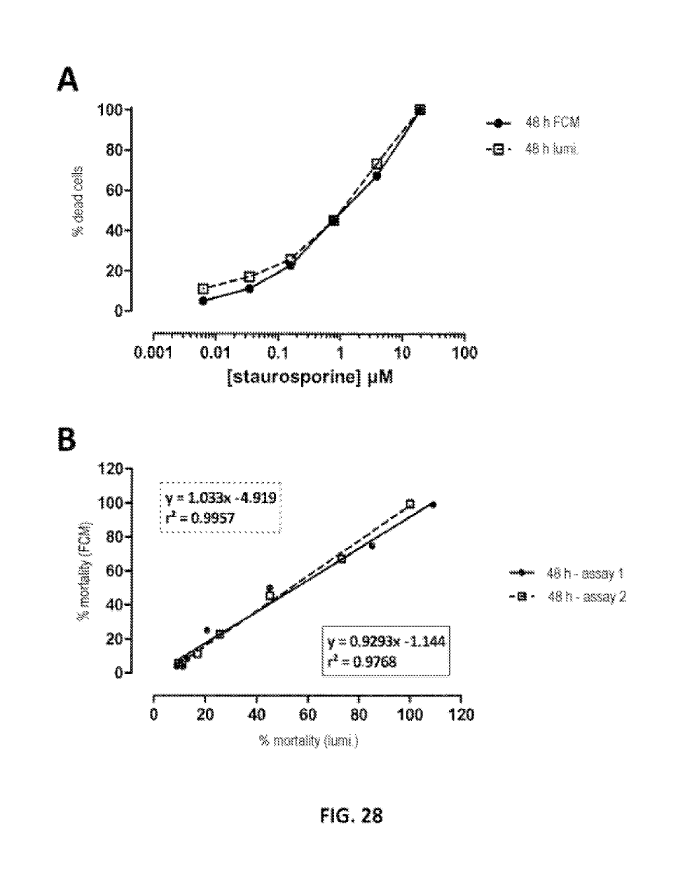

FIG. 28 illustrates the capacity of the nanoluciferase release method to measure the target-cell death induced by a pro-apoptotic treatment of chemical type.

### EXAMPLES

In the method according to the present invention described below, the target cells were permeabilized by a series of electric shocks (electroporation) in the presence of the two plasmids of interest presented in FIG. 5, one carrying the nanoluciferase transgene and the other the transgene of the enzyme allowing its integration into the genome, the PiggyBac transposase.

Legend of FIG. 5:
AmpR: ampicillin resistance gene;
pUC (ori): bacterial origin of replication;
Lac(F): gene encoding the lactose-specific enzyme III of S. aureus;
3′PB TR: 3′ homologous recombination element of the PiggyBac system;
Core insulator: chicken hypersensitivity site 4 core insulator;
SV40-polyA: Simian virus 40 Poly-Adenosine;
WPRE: Woodchuck hepatitis virus Posttranscriptional Regulatory Element;
PuroR: puromycin resistance gene;
EF1A: EF1A promoter;
Nanoluciferase: nanoluciferase gene;
CMV: CMV promoter;
5′PB TR: 5′ homologous recombination element of the PiggyBac system;
PiggyBac: PiggyBac gene.

Several target-cell lines underwent this genetic transformation: the Raji line, the SKOV3 line and a CHO line expressing human TNF-alpha.

After genetic transformation of the chosen target cells, said cells are placed in culture under suitable conditions (generally, for mammalian cells, the temperature is 37±2° C. in a humidified atmosphere containing between 5% and 10% of CO₂) in a culture medium suitable for the cell type chosen, containing the selectable antibiotic of which the resistance gene was introduced into the expression vector (puromycin in the example presented in FIG. 5). This antibiotic makes it possible to select the cells which are actually transformed by not allowing the growth of the cells for which the genetic transformation has not worked. A long culture (a few weeks) makes it possible to obtain a mixture of cells which have all stably integrated the transgene into their DNA.

Although this is not essential to the implementation of the present invention, it is recommended to subsequently carry out a step of cloning of these cell mixtures. This makes it possible to obtain a line of target cells expressing the nanoluciferase and derived from a single cell (or clone), which will promote the uniformity and the reproducibility of the results of the cytotoxicity test.

In order to guarantee the stability and the continuity of the supply of transformed target cells, it is preferable to produce a bank of these cells under conditions which allow their growth statically or with shaking in suitable containers. For example, to produce cell banks having made it possible to confirm the validity of the method of the present invention, the cells were cultured statically in flasks treated for cell culture, at 37±2° C. under a humid atmosphere at 5±1% of CO₂ and in suitable medium: RPMI-1640 containing 10% of fetal calf serum (FCS) and 0.25 g/ml of puromycin for the Raji line; in McCoy 5A medium, 10% FCS and 5 μg/ml of puromycin for the SKOV3 cells; in F12-Ham medium, 10% FCS, 1 mg/ml G418 and 20 μg/ml puromycin for the CHO cells. After growth of a sufficient amount of cells, the latter are dispensed into suitable containers and frozen according to the techniques which are customary for eukaryotic cells for the cryopreservation thereof.

FIG. 4 illustrates the type of signal that it is possible to obtain with such target cells (CHO cells expressing TNF-alpha in this example) expressing nanoluciferase after genetic transformation. These results show, firstly, that the signal generated by the nanoluciferase free in solution (after production by the cells) is perfectly linear between 10¹ and 10⁶ relative luminescence units (RLU). They show, secondly, that the ratio between maximum signal (after lysis of the cells with a detergent) and spontaneous release is always at least equal to 2 under the conditions tested, and stabilizes around 6 when the number of cells per well is between 250 and 5000 cells. The amount of target cells used in the assays presented below is thus included in this range. The conditions for use and the sensitivity of this system thus allow a great deal of flexibility in the implementation of the test. The very low number of cells required to obtain a satisfactory signal is particularly noteworthy when it is compared with the amount of cells required by the prior art techniques (see table 1 comparing the performance levels of the various methods of measurements specific for direct lysis [prior art and various examples of the invention described below with nanoluciferase]).

TABLE 1

| | | Required | | | | |
|---|---|---|---|---|---|---|
| | Signal/noise | amount | | | | High-throughput |
| Method | ratio | of cells | Radioactive | Sensitivity | Reproducibility | analysis capacity |
| ⁵¹Cr chromium | 5-12 | 2500 to | + | + | + | + |
| | | 10 000 | | | | |
| Calcein-AM | 2.5 | 2500 to | − | −/+ | −/+ | + |
| | | 10 000 | | | | |
| Eu^{3+/}Tb³⁺ | 1-3 | 5000 to | − | −/+ | −/+ | + |
| | | 10 000 | | | | |
| Flow | NA | 1000 to | − | + | −/+ | − |
| cytometry | | 5000 | | | | |
| Beta-galacto- | ≈30 | 2 × 10⁵ | − | −/+ | No data | + |
| sidase | | | | | available | |
| Nanoluciferase | 4-12 | 250 to | − | + | + | + |
| (present invention) | | 5000 | | | | |

Legend. “NA”: not applicable; “+”: yes; “−”: no; “−/+”: low.

Among the prior art methods: the current reference method is the ⁵¹Cr chromium method; the calcein-AM and Eu³⁺/Tb³⁺ methods are not very sensitive and are variable depending on the cell type, and the beta-galactosidase method underestimates by 30-40% the proportion of dead cells.

### Carrying Out the Cytotoxicity Test or Assay According to the Invention:

The target cells prepared as described above are placed in the form of a homogeneous suspension and counted by any appropriate method. The amount of cells required for carrying out the assay is deposited in a centrifugation tube and the culture medium is eliminated by centrifugation for between 1 and 30 minutes at an acceleration of from 100 g to 1500 g. This centrifugation is preferably carried out conventionally between 120 g and 600 g for 5 to 10 minutes, the supernatant culture medium is eliminated and the cells are taken up in new culture medium (preferably not containing the selectable antibiotic(s)) at the desired concentration for carrying out the final assay.

The cytotoxicity assay can be carried out in any type of container (tubes or plates) which make it possible to maintain eukaryotic cells alive for several hours, in particular 96-well or 384-well flat-, conical- or round-bottomed multiwell plates. Each test well contains a mixture including the chosen amount of target cells, the product of which the therapeutic/cytolytic action is to be tested and, if necessary, a complementary effector system such as cyototoxic cells (in the case of an assay of ADCC or mixed lymphocyte reaction type for example) or a source of complement (in the case of a CDC assay or the like). On the basis of the data presented in FIG. 4, the amount of target cells per well will be between 10 and 10 000, preferably between 200 and 3000.

The active substance is tested using various concentrations thereof, a single concentration per well. When an effector system (of effector cell or complement source type) is required, the substance to be tested can be added to the target cells either simultaneously with or before the addition of the effector system, while adhering to a variable pre-incubation time, preferably between 50 and 60 minutes. Several tubes or wells may be used with strictly similar conditions in order to perform several “replicates”.

In the case of an ADCC or mixed lymphocyte reaction assay, the ratio between the amounts of effector cells and of target cells is preferably between 0.1 and 200. The effector cells may be any cells capable of exerting a cytotoxic activity against the chosen cell line, such as NK cells, T lymphocytes, monocytes, macrophages or polymorphonuclear cells, which may be primary or immortalized cells, and non-modified or genetically transformed. This cytotoxic activity may be natural (that is to say may be triggered directly when target cells and effector cells come into contact) or may be induced by an activator substance, which may or may not be the substance to be tested (solutions of monoclonal or polyclonal antibodies or related molecules, pro-cytotoxic molecules, cytokines, hormones, neurotransmitters, etc.). The effector cells, in culture or originating from a vial from a cell bank that has been extemporaneously thawed, are thus counted by any appropriate method. The amount of cells required for carrying out the assay is then deposited in a centrifugation tube and the culture medium is eliminated by centrifugation for between 1 and 30 minutes at an acceleration of from 100 g to 1500 g, preferably between 120 g and 600 g for 5 to 10 minutes. The supernatant culture medium is then eliminated and the cells are taken up in new culture medium (preferably the same as for the target cells) at the desired concentration for carrying out the final assay. This concentration depends on the number of target cells used per condition and on the effector/target ratio chosen.

In the case where complement activation is desired, a source of complement is added to the tubes or wells. This source of complement may be an animal serum (for example from bovines, members of the sheep family, members of the goat family, rodents, rabbits, monkeys or human beings) or may consist of a mixture of the various purified and/or recombinant constituent molecules of the complement system. Since the complement system is very well conserved over evolution, the species from which the source of complement originates is of little importance. This experimental system is also suitable for testing the cytolytic effect of the serum in itself (without the addition of exogenous antibody), for example in processes for screening autoimmune or vaccine sera. Overall, the proportion of serum or of the source of complement may represent from 0.1% to 100% of the total reaction volume.

Alternatively, the test can be carried out using a single concentration of the cytotoxic substance to be tested, while varying the amount of effector system in the wells (effector/target ratio or amount of complement for example), or a mixture of the 2 approaches. Be that as it may, the principle of the assay remains the same.

After having mixed, in the tubes or the wells, the desired amounts of target cells, of substance(s) to be tested and/or of the effector system(s), the tubes or culture plates are incubated at between 34° C. and 40° C., preferably 37° C.±1° C., in an oven or an incubator. The incubation time can be variable depending on the process studied. In the case of phenomena such as ADCC or CDC, the incubation time will generally be between 1 hour and 8 hours, and preferably between 2 hours and 5 hours. In the case of apoptosis phenomena or cytotoxic effects in the long term, the duration of the assay can extend from 8 hours to 72 h, preferably between 24 h and 48 h.

At the end of the assay, the supernatant of each well is recovered. Preferably, and in order to be sure that cells will not be removed, the plates or the tubes containing the cells are centrifuged between 10 s and 30 min at an acceleration of from 100 g to 1500 g, preferably for between 30 and 120 s at a speed of between 200 g and 1000 g. Alternatively, an intermediate step of removing the supernatant directly from the wells or the reaction tubes followed by the transfer of this supernatant into a new plate or new tube(s) and the centrifugation thereof under the conditions above can be carried out. The volume of supernatant required for the reading is then transferred into a plate suitable for reading in a luminometer, that is to say opaque and white or black in color, and mixed with a nanoluciferase substrate. These substrates are of the family of coelenterazine and analogs thereof, and preferably of furimazine type commercially available from the company Promega under the product reference “Nano-Glo® Luciferase Assay Substrate”. Depending on the type of plate used, the supernatant is mixed with the substrate for a final reaction volume of 25 μl to 340 μl in standard 96-well plates, from 15 μl to 175 μl for “half-well” format 96-well plates, from 15 μl to 110 μl for standard 384-well plates, from 4 μl to 25 μl for “small volume” 384-well plates, or from 3 μl to 10 μl for 1536-well plates.

Depending on the recommendations of the supplier and on the results presented in FIG. 6, the “Nano-Glo® Luciferase Assay Substrate” is diluted by mixing with the supernatant (itself pure or diluted according to the expected strength of the signal and in a cell model-dependent manner) in a ratio of between 1/50 (1 volume of substrate per 50 volumes of supernatant) and 1/1000, and preferably between 1/100 and 1/200. After a minimum incubation time of 3 minutes, the plate is read in a luminometer (for example Mithras LB 940 model). The signal acquisition time is about 0.05 second.

As demonstrated in the examples presented below, the strength of the luminous signal is proportional to the lysis of the target cells. The signal generated is strong, resulting in a sensitive method, even with small amounts of target cells (starting from 250 cells per well). The examples below show that the method according to the invention based on measuring the release of nanoluciferase has characteristics and performance levels that are entirely comparable to those of the current reference test based on ⁵¹Cr release for the detection and measurement of cell death (see table 2 in example 10). On the other hand, it has the very large advantage of avoiding the use of radioactive elements, and thus all the constraints and all the risks associated with this use, while at the same time enabling experiments to be performed more rapidly since it does not require any target-cell labeling time. The principle of reading by means of a luminometer also constitutes a simple and economical method, very widespread in laboratories and not requiring the purchase of expensive equipment. Finally, its operating mechanism is entirely similar to that of the chromium test and the measurement is specific for target cell death, even when said target cells are mixed with other types of cells of which the death must not be measured.

All of these data demonstrate that the cytotoxicity test based on the release of nanoluciferase has all the characteristics required to become a reference test, in particular at the industrial level, for any assay requiring the specific measurement of actual death of a given cell population. In a nonrestricted manner, these tests could evaluate the cytotoxic effect of pollutants or contaminants, of environmental factors or of cytotoxic medicaments or drugs produced by chemical synthesis or by a biological system and of any other element that is toxic to a given cell population.

### Comparative Example A

Three different cell lines were labeled according to the method of the DELFIA BATDA kit (Perkin-Elmer), strictly according to the recommendations of the manufacturer. 10000 cells per well (3 wells per condition) are then incubated for 2 hours at 37° C. in culture medium alone (RPMI [Roswell Park Memorial Institute] containing 10% of fetal calf serum [FCS]) for determining the spontaneous release, or in culture medium containing 1% of Triton X-100 detergent for determining the maximum release (final volume of the wells=200 up. The background noise is determined on culture medium alone, with no cells and no Triton X-100. At the end of the incubation, 20 μl of supernatant are transferred into specific plates (supplied in the kit) and 200 μl of europium solution are added. After incubation for 15 minutes, the signals are read by the TRF method on a suitable reader (Mithras LB 940, Berthold Technologies, Thoiry, France). Presented in FIG. 1A are the absolute values measured for the various parameters in counts per second for the various lines. FIG. 1B represents the ratios of the maximum signal and the background noise (white bars) on the one hand, or the spontaneous signal (black bars) on the other hand. This figure shows the results of an experiment representative of approximately ten experiments during which various signal strength optimization approaches were attempted unsuccessfully, with signal/noise ratios always below 3.

### Comparative Example B

A first artificial reporter molecule was tested by the inventors, combining two distinct specific antigenic motifs (called flags), each recognized by an antibody carrying, for one of them, an energy-donating lanthanide and, for the other, an energy acceptor. The assembly represents a protein having a weight of 47 kDa. The presence of the released molecule is detected by adding the two antibodies simultaneously to the supernatant. If the free molecule is present, the two antibodies bind thereto, and are thus in direct proximity to one another, thereby allowing the transfer of energy and the emission of a signal of TR-FRET type.

In the example, SKBR3 target cells expressing this reporter molecule are incubated for 4 hours at 37° C. in a 96-well plate in the presence of increasing concentrations (indicated along the x-axis) of trastuzumab and of cytotoxic cells (T lymphocytes expressing the CD16 receptor). At the end of the 4 hours of incubation, the revealing antibodies are added to the medium and the TR-FRET signal is read by fluorimetry on a Mithras LB 940 reader (Berthold) (reading shift of 300 μs, ratio 665/620 nm).

In parallel, a fraction of the same SKBR3 cells expressing the reporter molecule is labeled with ⁵¹Cr (100 μCi/10⁶ cells). The remainder of the experimental conditions are strictly identical: number of target cells (3000) and effector cells (30 000) per well, culture medium (RPMI and 10% of fetal calf serum), two wells in duplicates for each concentration tested, 4 hours of incubation at 37° C. At the end of the incubation, the supernatant is recovered, transferred into scintillation plates (Lumaplate, Perkin-Elmer) and read in a scintillation counter (Microbeta Jet, Perkin-Elmer).

The results, presented in FIG. 2, are expressed as percentage of specific lysis, calculated according to the formula:

$$\frac{Signal ( X ) - Spontaneous signal}{Maximum signal - Spontaneous signal}$$

wherein “Signal (X)” is the signal obtained for a given well X, “Maximum signal” is the signal obtained when the cells are lysed with 0.75% of triton X-100 and “Spontaneous signal” is the signal obtained when the cells are simply incubated in the presence of culture medium alone (with no antibodies and no effector cells). The graph shows, along the y-axis, the mean specific lysis ±standard deviation for each concentration tested according to the two methods, by ⁵¹Cr (black circles and solid line) or TR-FRET (black triangles and dashed line). The results presented in FIG. 2 relate to one experiment representative of two.

These results show the ineffectiveness of the reporter molecule in terms of being representative of the target-cell lysis as observed by the ⁵¹Cr method. Since the positive controls (not shown) are moreover satisfactory, the detection method in itself is not to be called into question. The reporter molecule is thus probably retained in the cytoplasm of the target cells and is not released during the lysis. The large size of the molecule (47 kDa) is the preferred hypothesis for explaining this cytoplasmic retention.

### Comparative Example C

Another reporter protein, a recombinant GFP (Green Fluorescent Protein) of smaller size than that presented in comparative example B above (approximately 27 kDa), and the presence of which in the supernatant can be measured using an antibody coupled to a lanthanide (which performs a TR-FRET energy transfer to the GFP when it is bound thereto), was tested in this comparative example.

An SKOV3 cell target line genetically transformed to express recombinant GFP was used for these assays, the results of which are presented in FIG. 3. The left-hand part (A) of FIG. 3 shows the GFP expression measured by flow cytometry on the non-transformed wild-type (wt) SKOV3 cells, at the top, or by the transformed (GFP-positive) SKOV3 cells, at the bottom. The GFP expression by the transformed cells corresponds approximately to 1.5 Log, which is a satisfactory level.

The right-hand part of FIG. 3 (B) represents the assay in which 30 000 or 200 000 cells were deposited in triplicate in wells of a 96-well plate and incubated in the presence of culture medium alone (RPMI containing 10% of FCS) (spontaneous release) or of culture medium plus 0.75% of triton X-100 (maximum release). At the end of incubation, the supernatant is removed and an anti-GFP antibody coupled to a lanthanide (Tb) is added. The signal measured (Mithras LB 940 reader, Berthold Technologies) is a TR-FRET signal from the lanthanide to the GFP (reading shift=300 μs, ratio 520/620 nm). FIG. 3B represents the ratio of the maximum signal to the spontaneous signal for each of the two amounts of cells, and is representative of one experiment among two.

The results show that, even with a large amount of target cells per well (3×10⁴), the maximum-to-spontaneous ratio is only 2, and that a considerable amount of cells (2×10⁵) is required in order to begin to observe an advantageous ratio (of about 4). This method is thus very far from the performance levels of ⁵¹Cr in terms of sensitivity (as mentioned above, said sensitivity is between 8 and 12 with 3000 cells per well). Here again, the size of the GFP and/or the mechanisms of its release during cell lysis are not compatible with an effective measurement of cell death.

### Example 1: Shows the Linearity of the Luminescent Signal and the Influence of the Amount of Cells on this Signal

The luminescence of a supernatant containing nanoluciferase was measured, after lysis with triton X-100, serially diluted between 1/1 and 1/10 000. 25 μl of each dilution are mixed with 25 μl of Nano-Glo® substrate (Promega) in a “half-well” 96-well plate before reading on a luminometer (Mithras LB 940, Berthold Technologies, acquisition time 0.05 s). A linear regression of the values obtained is performed (see FIG. 4A: strength of the luminescent signal obtained, expressed in RLU (Relative Luminescence Unit) as a function of the dilution). It is noted, firstly, that this signal is linear (regression coefficient R²=0.9997) over the entire measurement range and extends approximately from 10 to 10⁶ arbitrary luminescence units on the reader used.

Secondly, the linearity of the signals from spontaneous and maximum release of CHO-K1 cells expressing nanoluciferase was evaluated by incubating for 4 h variable amounts of target cells (10 000, 5000, 2500, 1000, 500, 250, 100, 50 and 25 cells per well) in the presence of medium alone (spontaneous) or of 0.25% of triton X-100 (maximum). 25 μl of supernatant are then removed and mixed with 25 μl of Nano-Glo® substrate before reading with a luminometer (acquisition time 0.05 s). The results are presented in FIG. 4B. The x-axis shows the number of cells per well and the y-axis on the left represents the luminescent signal obtained under the spontaneous (black circles) and maximum (black squares) conditions. A linear regression is performed for each of these two series of points and represented on the graph (respectively large dashed line and continuous line), and also the associated correlation factor (R²) which shows a very good linear relationship between these parameters. For each amount of cells, the ratio of maximum signal to spontaneous signal is calculated and represented by a white triangle on the right-hand y-axis, each point being connected by a thin dashed line to facilitate reading.

All of these data demonstrate the feasibility of this method, which proves to be sensitive (25 cells per well are sufficient to have a satisfactory signal), linear over a wide range of cell amounts and of luminescence (all the regression factors analyzed are greater than 0.94) and flexible to use (the [maximum release]/[spontaneous release] ratio remains constant between 250 and 2500 cells per well).

### Example 2: Shows the Linear Relationship Between the Strength of the Luminescent Signal and the Number of Cells Actually Dead During a Cytotoxicity Assay

A complement lysis assay was carried out on Raji cells (expressing nanoluciferase) in the presence of a commercially available anti-CD20 therapeutic antibody, MabThera® (INN: rituximab).

These Raji cells expressing nanoluciferase are incubated for 4 h at 37° C. in flat-bottomed 96-well plates (3000 cells per well) in RPMI-1640 medium, in the presence of variable amounts of anti-CD20 antibodies (MabThera®, Roche, range of 9 concentrations from 1093.5 to 0.167 ng/ml per 1/3 dilution increment) and of 1 CH₅₀ (hemolytic complement 50, usual unit of measurement of complement activity) of guinea pig complement (Sigma-Aldrich). Control conditions are also carried out, making it possible to measure the spontaneous release (3000 target cells in RPMI-1640, 1 CH₅₀ of guinea pig complement and 1093.5 ng/ml of an antibody which does not bind to Raji cells, Herceptin®) and the maximum release of nanoluciferase in the presence of 0.01% of triton X-100. Each operating condition is carried out in triplicate. At the end of the incubation, 25 μl of supernatant are recovered from each well, mixed with 25 μl of Nano-Glo® substrate diluted to 1/50 in PBS, incubated for 3 min and read with a luminometer.

The percentage of specific lysis obtained for each concentration X is calculated from the luminescence measurements (in RLU) according to the formula: (RLU(X)−RLU(spontaneous))/(RLU(max)−RLU(spontaneous))×100.

After removal of the supernatant, the remainder of the cells is resuspended, extemporaneously mixed with 0.25 μM final concentration of the TO-PRO-3 vital label (Life Technologies) and analyzed by flow cytometry in order to determine the percentage mortality (that is to say the percentage of TO-PRO-3-labeled cells). FIG. 7 presents the results obtained for each of the MabThera® concentrations. Each point represents the mean of the triplicates, the vertical and horizontal bars representing the standard deviation. The theoretical line y=x is represented by a dashed line. The results show that the regression line has a slope of 1.08±0.10, an ordonate at the origin of 3.01±4.21 and a linear regression coefficient of 0.8242. These results show a satisfactory proportional and linear relationship between the two methods of measuring cytolysis, which makes it possible to confirm that the lysis phenomenon measured by the nanoluciferase method is functionally well reflected by an actual death of the cells at the biological level.

### Example 3

FIG. 8, in which the specific lysis of adherent cells (SKOV3) or non-adherent cells (Raji) expressing nanoluciferase was measured in an ADCC assay according to the present invention, shows the dynamic nature of said method.

Non-adherent (Raji) or adherent (SKOV3) target cells transformed to stably express nanoluciferase were incubated (at 37° C. in a humidified atmosphere at 5% CO₂) in flat-bottomed 96-well plates treated for cell culture (3000 target cells per well), in the presence of increasing concentrations (the logarithms of which are indicated along the x-axis) of the corresponding antibody (rituximab [MabThera®] or trastuzumab [Herceptin®], respectively) and of cytotoxic effector cells (T lymphocytes expressing CD16, 30 000 effector cells per well) in RPMI-1640 supplemented with 5% of FCS. Each range is deposited 4 times (for the 4 incubation times tested) with 3 wells per condition (triplicate). After 1 (black circles), 2 (black squares), 3 (white diamonds) or 4 (black triangles) hours of incubation, 25 μl of supernatant are recovered from the corresponding wells, transferred into a white “half-well” plate, mixed with 25 μl of NanoGlo® substrate diluted to 1/50 in D-PBS, incubated for 3 minutes and read on a Mithras LB940 luminometer. The percentage of specific lysis obtained for each concentration X is calculated from the luminescence measurements (in RLU) according to the formula (RLU(X)−RLU(spontaneous))/(RLU(max)−RLU(spontaneous))×100, the maximum and spontaneous conditions being obtained by incubation of the same cells in a medium containing an irrelevant antibody (3000 ng/ml of trastuzumab or 5000 ng/ml of rituximab, respectively, to constitute the spontaneous release measurement) and 0.01% of triton X-100 (maximum release). The percentages of specific lysis are represented along the y-axis, each point representing the mean of the triplicate and the vertical bars representing its standard deviation. The data were then modeled by means of a 4-parameter sigmoidal regression using the GraphPad Prism software.

A gradual increase in the amount of lysed cells, concomitant with, on the one hand, the increase in the concentration of cytolytic agent (the therapeutic antibody) and with, on the other hand, the incubation time, is observed. In the two models, 1 and 2 hours are times that are too short to observe the maximum lytic effect, whereas 3 and 4 hours show a saturation of the cytotoxicity phenomenon. In the latter case, an incubation time longer than 3 h does not make it possible to obtain a higher lysis of the target cells. The present method is thus also capable of evaluating the kinetic aspects of the cytolysis.

### Example 4

In order to evaluate their performance levels under strictly comparable conditions, the ⁵¹Cr and nanoluciferase methods were compared in parallel in one and the same ADCC assay specific for the CD20 molecule, repeated 3 times independently (one of the assays being carried out by a second operator). The same Raji cells expressing nanoluciferase were or were not labeled with ⁵¹Cr and were used in parallel in the assay.

Raji target cells transformed to stably express nanoluciferase were incubated or not incubated for 1 hour in a saline solution of Na₂CrO₄ equivalent to 100 μCi of ⁵¹Cr per million cells, then washed 4 times with RPMI-1640, 10% FCS, and counted. The cells (3000 per well), labeled or not labeled with ⁵¹Cr, were then incubated at 37° C. in a humidified atmosphere at 5% CO₂ in flat-bottomed 96-well plates treated for cell culture, in the presence of increasing concentrations of the rituximab antibody (MabThera®) and of cytotoxic effector cells (T lymphocytes expressing CD16, 30 000 cells per well) in RPMI-1640 supplemented with 5% of FCS.

After 4 hours of incubation, the percentage of specific lysis is measured according to the corresponding method (release of ⁵¹Cr or of nanoluciferase depending on whether the cells have or have not been labeled with chromium): 25 μl of supernatant of the ⁵¹Cr-labeled cells are recovered and deposited on a Lumaplate® (Perkin Elmer). At the same time, for the cells not labeled with ⁵¹Cr, 25 μl of supernatant are recovered from each well, transferred into white “half-well” 96-well plates, mixed with 25 μl of NanoGlo® substrate diluted to 1/50 in D-PBS, incubated for 3 minutes and read on a Mithras LB940 luminometer (Berthold Technologies). After drying overnight, the radioactivity of the supernatants deposited in the Lumaplate® is measured in a Microbeta-Jet counter (Perkin Elmer) and expressed in CCPM (corrected count per minute) after standardization of the detectors.

The percentage of specific lysis obtained for each concentration X is calculated in the same way for the two types of methods, from the measurements of radioactivity (in CCPM) or of luminescence (in RLU) according to the formula

$$\frac{CCPM / RLU [ X ] - CCPM / RLU [ spontaneous ]}{CCPM / RLU [ max ] - CCPM / RLU [ spontaneous ]} \times 100.$$

In both cases, the maximum and spontaneous conditions were obtained by incubation of the same cells in a medium containing 3000 ng/ml of trastuzumab as a replacement for rituximab (spontaneous release) and 0.01% of triton X-100 (in the case of the maximum release only). The percentages of specific lysis are represented in FIG. 9 along the y-axis, each point representing the mean of the nine determinations (3 replicates in 3 independent experiments) and the vertical bars representing the standard deviations. The data were modeled by means of a 4-parameter sigmoidal regression using the GraphPad Prism software.

This modeling makes it possible to calculate four characteristic parameters of the curve: the minimum (Emin) and maximum (Emax) lysis percentages, the slope of the linear part and the antibody concentration required to obtain 50% of the Emax (EC₅₀). The capacity of the two methods to detect samples of which the ADCC activity is slightly modified compared with a reference sample (which constitutes the basis of a measurement of potency) was evaluated by means of the use of ranges of antibodies of which the concentrations are shifted by 80% or by 125% relative to the reference range (termed “100%”). The potency of the ranges diluted to X % is calculated according to the formula: (EC₅₀[100%])/(EC₅₀[X %])×100. This approach also makes it possible to evaluate the accuracy of the method according to the ICH-Q2(R1) criteria.

The general results of these three experiments are illustrated in FIG. 9, which shows the percentages of specific lysis obtained (mean±standard deviation of the 3 experiments) and the resulting modelings for the 100% range. They show a very good reproducibility of the two methods (the standard deviations for one and the same condition are not very high) and also a results profile that is very similar between the two methods (the two curves are virtually superimposable).

To push the analysis further, the dispersion of the Emin and Emax calculated for each series of data, for the 3 ranges and in the 3 experiments, was analyzed. The results are presented in FIG. 10 (where each point corresponds to the value measured for one experiment, the horizontal line for each series of values representing the arithmetic mean of the series in question). The dispersions are entirely satisfactory, with mean values (Emin and Emax, ±standard deviation) of 1.0±1.5% and 75.7±6.0% for ⁵¹Cr and of 1.2±1.2% and 73.6±8.4% for nanoluciferase. The results show that Emin and Emax are constant and reproducible between the assays, even when 80% and 125% concentration ranges are used in addition to the standard range.

In the same way, the analysis of the dispersion of the EC₅₀ values of the 100% ranges [FIG. 11: each point represents the EC₅₀ value obtained in an experiment using either the measurement of the nanoluciferase released (black circles) or that of the chromium-51 (white squares), the horizontal line representing the arithmetic mean of the 3 values] shows results that are very satisfactory for the two methods and a mean value that is slightly lower for the luminescence method: 55.2±15.6 ng/ml for ⁵¹Cr and 31.8±7.3 ng/ml for nanoluciferase (means±standard deviations), that is to say coefficients of variation (CV) of 28.3% and of 22.8% respectively.

Finally, the potency is calculated for each independent experiment as the ratio (expressed as percentages) of the EC₅₀ of the reference sample (in this case the 100% standard range) to the EC₅₀ of the sample to be tested (in this case the 80% and 125% ranges). The results are represented in FIG. 12: the potency calculated is reported along the y-axis for the two methods, nanoluciferase (black circles) and chromium-51 (white squares), the horizontal line (solid for the nanoluciferase method and thick dashed for the ⁵¹Cr method) represents the arithmetic mean of the 3 measurements. The thin dashed lines indicate the ideal theoretical potencies of 80% and 125%.

The results show here again a comparable dispersion of the measurements between the two methods and results which are in accordance with those expected. Indeed, the mean values (±standard deviation) for the 80% and 125% ranges are 76.7±10% (CV=14.2%) and 131.9±11.9% (CV=9.1%) for the ⁵¹Cr method and 83.6±22% (CV=26.4%) and 119.5±22.7% (CV=19.0%) for the nanoluciferase method.

These results demonstrate the relevance of the method based on the release of nanoluciferase in the anti-CD20 ADCC model described here, the performance levels of which are entirely comparable to those of the ⁵¹Cr reference method.

### Example 5

Using the same type of target cells as that of example 4, the nanoluciferase method was compared to the ⁵¹Cr method in an anti-CD20 CDC assay. The methodology applied was the same as that described in example 4, the cytolytic system being formed by a source of complement. In the same way, a standard range of antibody (rituximab) was used, and also 80% and 125% modified concentration ranges. Three independent experiments were carried out, one of which was carried out by a second operator.

FIG. 13 shows the comparison of the sigmoid curves obtained from the mean values resulting from the 3 experiments. The standard deviations are satisfactory and the curves are entirely similar in terms of their appearance, despite a slight shift in the EC₅₀ values.

Fine analysis of the Eₘᵢₙ and of the Eₘₐₓ values (FIG. 14) confirms the similarity of these parameters, with 3.0±3.4% and 88.6±7.8% for ⁵¹Cr and −0.3±3.2% and 95.6±7.0% for nanoluciferase, respectively (means±standard deviation) for all of the 80%, 100% and 125% ranges.

Analysis of the dispersion of the EC₅₀ values for the 100% range in the 3 experiments (FIG. 15) itself also gives satisfactory results with a mean value (±standard deviation) of 156.7±30.1 ng/ml (CV=19.2%) for ⁵¹Cr and of 73.1±9.9 ng/ml (CV=13.5%) for nanoluciferase.

Finally, calculation of the potencies (FIG. 16) makes it possible to note similar performance levels between the two methods, with values (means±standard deviations) that are entirely comparable for the 80% range (69.6±12.3% for ⁵¹Cr and 65.0±12.9% for nanoluciferase, CV=17.7% and 19.8% respectively) and better precision with the 125% range for the nanoluciferase method despite a slightly worse accuracy (105.8±8.2% [CV=7.7%], compared with 131.5±16.4% [CV=12.4%] for 51Cr).

These results demonstrate the relevance of the method based on the release of nanoluciferase in the anti-CD20 CDC model described here, the performance levels of which are entirely comparable to those of the ⁵¹Cr reference method.

### Example 6

The nanoluciferase method was compared to the ⁵¹Cr method in an anti-HER2 ADCC assay. For this, SKOV3 cells stably expressing nanoluciferase were or were not labeled with ⁵¹Cr and were used simultaneously in an ADCC assay under the same experimental conditions, according to the same principle as that described in example 4.

SKOV3 target cells transformed to stably express nanoluciferase were incubated or not incubated for 1 hour in a saline solution of Na₂CrO₄ (Perkin Elmer, France), equivalent to 100 μCi of ⁵¹Cr per million cells, then washed 4 times with RPMI-1640, 10% FCS and counted. The cells (3000 per well), labeled or not labeled with ⁵¹Cr, were then incubated at 37° C. in a humidified atmosphere at 5% CO₂ in flat-bottomed 96-well plates treated for cell culture, in the presence of increasing concentrations (1, 5, 10, 25, 50, 100, 250, 500, 1000 and 5000 ng/ml, the logarithms of which are indicated along the x-axis) of the trastuzumab (Herceptin®) antibody and of cytotoxic effector cells (T lymphocytes expressing CD16, 30 000 cells per well) in RPMI-1640 supplemented with 5% of FCS.

At the end of the 4-hour incubation, 25 μl of supernatant of each condition are recovered, the cytolysis is measured and the percentage of specific lysis is calculated according to modes strictly identical to those described in example 4.

A standard range (100%) of trastuzumab and also variations at 80% and 125% of this range were used, and the experiment was repeated 3 times independently, one of which being carried out by a second operator. The mean percentages of specific lysis obtained by the two methods in the three experiments and also the associated modelings are shown in FIG. 17. Here again, the two methods give very similar results, with a dispersion of the values which seems to be less for the nanoluciferase method.

These data are confirmed by the minimum and maximum lysis analysis (FIG. 18: each point corresponds to the value measured for one experiment, the horizontal line for each series of values representing the arithmetic mean of the series in question), which shows Eₘᵢₙ and Eₘₐₓ values (means±standard deviations) of 0.5±2.2% and 41.3±4.3% for ⁵¹Cr and of 0.3±0.7% and 34.1±4.3% for nanoluciferase, respectively.

The analysis of the EC₅₀ values (FIG. 19: each point represents the EC₅₀ value obtained in one experiment, the horizontal line representing the arithmetic mean of the 3 values) confirms the lowest dispersion obtained in this model by virtue of the nanoluciferase method. Thus, the mean EC₅₀ (±standard deviation) for the standard range is 181.6±8.0 ng/ml using nanoluciferase (CV=4.4%), whereas it is 402.5±172.5 ng/ml for ⁵¹Cr, that is to say a CV of 42.8%.

Despite this higher dispersion of the EC₅₀ values between the experiments for the chromium method, the analysis of the potencies (which is calculated experiment by experiment by dividing the EC₅₀ of the standard range by that of the modified range) does not show any significant difference in performance level between the two methods (FIG. 20). In this FIG. 20, the horizontal line (solid for the nanoluciferase method and thick dashed for the ⁵¹Cr method) represents the arithmetic mean of the three measurements. The thin dashed lines indicate the ideal theoretical potencies of 80% and 125%. The mean potency (±standard deviation) calculated for the 80% and 125% ranges is 82.9±13.0% (CV=15.7%) and 154.2±24.8% (CV=16.1%) for the ⁵¹Cr method and 69.3±7.9% (CV=11.4%) and 136.9±19.4% (CV=14.2%) for the nanoluciferase method, respectively.

All of these results demonstrate the usefulness of the nanoluciferase method as a replacement for the ⁵¹Cr method in this anti-HER2 ADCC model.

### Example 7

The method for measuring nanoluciferase release was compared to the chromium method in a third model, that of the anti-TNF-alpha antibody adalimumab (Humira®). The present example illustrates the implementation thereof in an assay for measuring the CDC activity. For this, CHO cells pretransformed and selected to stably express human TNF-alpha in its membraned form were transformed to stably express nanoluciferase. The methodology followed is then comparable to that described in example 5. Briefly, these cells, labeled or not labeled with ⁵¹Cr, were incubated in the presence of increasing concentrations of adalimumab (Humira®) and of a source of complement (from guinea pig (Sigma)). The specific lysis was then measured by the appropriate method (chromium release or nanoluciferase release).

The percentage of specific lysis obtained for each concentration X is calculated in the same way for the two types of methods, on the basis of the measurements of radioactivity (in CCPM) or of luminescence (in RLU) according to the formula

$$\frac{CCPM / RLU [ X ] - CCPM / RLU [ spontaneous ]}{CCPM / RLU [ max ] - CCPM / RLU [ spontaneous ]} \times 100.$$

In both cases, the maximum and spontaneous conditions were obtained by incubating the cells in the same medium containing 8000 ng/ml of trastuzumab as a replacement for the adalimumab (spontaneous release) and 0.01% of triton X-100 (in the case of the maximum release only). The percentages of specific lysis are represented along the y-axis, each point (black circle for nanoluciferase and white squares for ⁵¹Cr) representing the mean of the nine determinations (3 replicates in 3 independent experiments) and the vertical bars representing the standard deviations. The data were modeled by means of a 4-parameter sigmoidal regression using the GraphPad Prism software.

The mean results obtained for the 3 experiments are represented in FIG. 21. They show here again a very good comparability between the two methods, with curves having very similar appearances and characteristics (plateaus, slopes, EC50).

The analysis of the Eₘᵢₙ and Eₘₐₓ dispersion carried out confirms this similarity (FIG. 22) with respective mean values (±standard deviation, over the whole of the ranges in the 3 experiments) of 3.2±2.8% and 50.4±5.6% for ⁵¹Cr and of 0.9±1.5% and 45.7±5.1% for nanoluciferase.

The analysis of the dispersion of the EC₅₀ values of the three CDC experiments described in FIG. 21 (FIG. 23) for the standard ranges gives in this case a virtually perfect equality between the two methods, the mean values (±standard deviation, mean represented by a horizontal line) of the 3 experiments being 150.8±15 ng/ml for chromium (CV=9.9%) and 156.9±23.4 ng/ml for nanoluciferase (CV=14.9%).

Finally, the analysis of the potencies through the 80% and 125% ranges itself also shows comparable performance levels between the two methods (FIG. 24) with mean potencies (±standard deviation) of 91.2±23.6% (CV=25.8%) and 153.9±29.2% (CV=19.0%) for ⁵¹Cr and of 83.4±10.1% (CV=12.1%) and 131.7±47.2% (CV=35.8%) for nanoluciferase (respectively for the 80% and 125% ranges).

These results demonstrate that the method based on measuring nanoluciferase release is equivalent in terms of results and performance levels to the ⁵¹Cr release method for measuring the CDC activity in an anti-TNF-alpha model.

### Example 8

The nanoluciferase method was compared to the ⁵¹Cr method in an anti-TNF-alpha ADCC assay. For this, the CHO cells described in example 7, stably expressing TNF-alpha and nanoluciferase, were used, as was a standard range of adalimumab and of variations at 80% and 125% of this range. Three independent experiments were carried out, according to the same methodology as in the previous examples. On the basis of the EC₅₀ values generated in the sigmoidal regression models using the specific lysis data, the potencies of the 80% and 125% ranges were calculated as previously and the results are presented in FIG. 25.

The results are comparable between the 2 methods, with a slight advantage in terms of dispersion of the values for the nanoluciferase method. Indeed, the mean potencies (±standard deviations) of the 80% and 125% ranges calculated are 91.4±43.3% (CV=47.4%) and 138.5±43.9% (CV=31.7%) for the ⁵¹Cr method and 75.5±15.5% (CV=20.6%) and 114.2±22.8% (CV=19.9%) for nanoluciferase. These results demonstrate the usefulness of the nanoluciferase method for measuring the ADCC activity of anti-TNF-alpha antibodies as a replacement for the ⁵¹Cr method.

### Example 9

This example illustrates the appropriate nature of the nanoluciferase method with respect to the biological phenomena involved in the ADCC and CDC mechanisms. For this, ADCC and CDC reactions were carried out in parallel and under the same experimental conditions using, for the incubation, either flat-bottomed plates or round-bottomed plates, the latter promoting cell contacts and interactions through the particular shape of the well. The results are presented in FIG. 26.

They show that, in the case of the ADCC assay for which the interactions between effector and target cells are essential to the cytotoxic effect, the shape of the well actually influences the maximum lysis percentage (38.1% on average for the round bottoms compared with 27.6% for the flat bottoms), without this phenomenon significantly modifying the resulting EC₅₀ values (59.8 ng/ml compared with 65.3 ng/ml, respectively). Conversely, the shape of the well does not influence the CDC reaction, with mean Eₘₐₓ values (47.0% in round bottoms and 44.6% in flat bottoms) and mean EC₅₀ values (114.1 and 155.3 ng/ml, respectively) which are similar and independent of the shape of the well.

Promoting contacts between the cells through a suitable well shape thus clearly makes it possible to improve the amount of target cells lysed by ADCC and detected by the nanoluciferase method. In a coherent manner, the CDC assay, in which the only cell population present is the target cell population and in which the effector system is soluble (the complement), is for its part insensitive to the shape of the well.

### Example 10

In order to compare the performance levels of the nanoluciferase release method with those of the ⁵¹Cr release reference method, the variability results presented in examples 4 to 8 were collated in table 2 below.

TABLE 2

| | | CV on | CV on 80% potency | | CV on 125% potency | |
|---|---|---|---|---|---|---|
| Model | Assay | EC50 | (and relative bias) | | (and relative bias) | |
| | | ⁵¹Cr | | | | |
| CD20 | ADCC | 28.3% | 14.2% | (−4.1%) | 26.4% | (+4.5%) |
| | CDC | 19.2% | 17.7% | (−13.0%) | 12.4% | (+5.2%) |
| HER2 | ADCC | 42.8% | 15.7% | (+3.6%) | 16.1% | (+23.4%) |
| TNF- | ADCC | 19.9% | 47.4% | (+14.3%) | 31.7% | (+10.8%) |
| alpha | CDC | 9.9% | 25.8% | (+14.0%) | 19.0% | (+23.1%) |
| | | nanoluciferase | | | | |
| CD20 | ADCC | 22.8% | 9.1% | (+5.5%) | 19.0% | (−4.4%) |
| | CDC | 13.5% | 19.8% | (−18.8%) | 7.7% | (−15.4%) |
| HER2 | ADCC | 4.4% | 11.4% | (−13.4%) | 14.2% | (+9.5%) |
| TNF- | ADCC | 14.5% | 20.6% | (−5.6%) | 19.9% | (−8.6%) |
| alpha | CDC | 14.9% | 12.1% | (+4.3%) | 35.8% | (+5.4%) |

All the results are expressed as percentages. Three independent experiments were carried out in each of the models and assays. The coefficients of variation (CV) of the EC₅₀ values and potencies are calculated as the standard deviation divided by the mean of the corresponding data. The relative bias expresses the deviation between the expected value and that actually obtained, as a percentage of the expected value, according to the following formula (where P represents the potency):

$$Relative bias = \frac{Mean P - theoretical P}{Theoretical P} \times 100$$

The objective of examples 11 and 12 below is to evaluate the capacity of the present method to measure cell death phenomena with kinetics longer than those involved in the ADCC or CDC mechanisms, for example in the case of the detection of the cytotoxic effect of pollutant, toxic or pro-apoptotic molecules.

### Example 11: Physical Agent

Raji cells transformed to stably express nanoluciferase (50 000 cells per well) are cultured for 48 hours at 37° C. (under a humidified atmosphere containing 5% CO₂) in RPMI-1640 medium containing 10% of FCS, and in the presence or absence of 0.2% of triton X-100 (to determine the maximum release), after having been exposed to ultraviolet (UV) radiation for a variable time (0, 10, 20, 30, 40, 50 or 60 seconds). After 48 h, 25 μl of supernatant are recovered from each well, transferred into a white “half-well” plate, mixed with 25 μl of NanoGlo® substrate diluted to 1/50 in D-PBS, incubated for 3 minutes and read on a Mithras LB940 luminometer (Berthold Technologies). The results of the reading are expressed in RLU (relative luminescence units). The percentage of dead cells for each concentration of staurosporine is determined according to the formula (RLU[triton-free medium])/(RLU[0.2% triton])×100.

After removal of the supernatant, the remainder of the cells are resuspended, extemporaneously mixed with a final concentration of 0.25 μM of the vital marker TO-PRO-3 (Life Technologies) and acquired on a C6 flow cytometer (BD/Accuri). The percentage of dead cells is determined during the analysis of the cytometry data as the percentage of cells having integrated the TO-PRO-3.

Two independent identical experiments were carried out, the results of which are presented on graphs A and B of FIG. 27. The graph at the top (A) represents the percentages of dead cells determined according to the 2 methods (nanoluciferase luminescence [“lumi.”, dashed line] or flow cytometry [“FCM”, solid line]) as a function of the UV-exposure time for a representative assay. The graph at the bottom (B) represents the percentage of dead cells determined by the luminescent method (“% mortality lumi.”) as a function of the corresponding percentage of dead cells measured by flow cytometry (“% mortality FCM”) for all of the conditions in assay 1 (solid line, solid circles) and in assay 2 (dashed line, open squares). A linear regression was calculated between these two parameters by means of the GraphPad Prism software and plotted on the graph (the equation of the straight line and the linear regression coefficient R² are represented on the graph for each of the 2 assays).

### Example 12: Chemical Agent

Raji cells transformed to stably express nanoluciferase (50 000 cells per well) are cultured for 48 hours at 37° C. (under a humidified atmosphere containing 5% CO₂) in the presence of increasing concentrations of staurosporine, which is a pro-apoptotic drug, in RPMI-1640 medium containing 10% of FCS, and in the presence or absence of 0.2% of triton X-100 (to determine the maximum release). At the end of the incubation, 25 μl of supernatant are recovered from each well, transferred into a white “half-well” plate, mixed with 25 μl of NanoGlo® substrate diluted to 1/50 in D-PBS, incubated for 3 minutes and read on a Mithras LB940 luminometer. The results of the reading are expressed in RLU (relative luminescence units). The percentage of dead cells for each concentration of staurosporine is determined according to the formula (RLU[triton-free medium])/(RLU[0.2% triton])×100.

After removing the supernatant, the remainder of the cells are resuspended, extemporaneously mixed with a final concentration of 0.25 μM of the vital marker TO-PRO-3 (Life Technologies) and acquired on a C6 flow cytometer (BD/Accuri). The percentage of dead cells is determined during the analysis of the cytometry data as the percentage of cells having integrated the TO-PRO-3.

Two identical independent experiments were carried out, the results of which are presented on graphs A and B of FIG. 28. The graph at the top (A) represents the percentages of dead cells determined according to the 2 methods (nanoluciferase luminescence [“lumi.”, dashed line] or flow cytometry [“FCM”, solid line]) as a function of the staurosporine concentration in the presence of which the cells were incubated, for a representative assay. The graph at the bottom (B) represents the percentage of dead cells determined by the luminescent method (“% mortality lumi.”) as a function of the corresponding percentage of dead cells measured by flow cytometry (“% mortality FCM”) for all of the conditions in assay 1 (solid line, solid circles) and in assay 2 (dashed line, open squares). A linear regression was calculated between these two parameters by means of the GraphPad Prism software and plotted on the graph (the equation of the straight line and the linear regression coefficient R2 are represented on the graph for each of the 2 assays).

These results show, firstly, that there is clearly a proportional relationship between the concentration of staurosporine and the mortality of the target cells, regardless of the method for measuring the mortality. Secondly, the mortality detected by the nanoluciferase measurement correlates perfectly with the analysis at the cell level by flow cytometry. Indeed, the slope of the regression line is close to 1 (slope=0.86), as is the linear regression coefficient (R²=0.9659).

Similar results (not represented) were obtained with the SKOV3 and CHO-TNF-alpha target lines expressing nanoluciferase.

1. A non-radioactive method for the direct in vitro determination (control and quantification) of the cytolytic action of an active agent with respect to target cells and/or of a medium surrounding target cells, comprising the successive steps below:
i) genetically transforming target cells to express an enzyme exogenous to said target cells,
ii) exposing said genetically transformed target cells to the active agent and/or to said surrounding medium to be tested, which can result in the lysis of at least one portion of the target cells while releasing said exogenous enzyme into the extracellular medium,
iii) measuring the activity of the exogenous enzyme released during the lysis of said target cells,
wherein said exogenous enzyme is an enzyme which has a molar mass of less than or equal to 45 kDa, and the activity of which can be detected by luminescence or fluorescence.

2. The method as claimed in claim 1, wherein the molar mass of the exogenous enzyme is less than or equal to 30 kDa, preferably less than or equal to 25 kDa, more preferably less than or equal to 20 kDa.

3. The method as claimed in claim 1, wherein said exogenous enzyme is a luciferase.

4. The method as claimed in claim 1, wherein said exogenous enzyme has a peptide sequence which exhibits at least 60% homology, preferably at least 80% homology, with the wild-type peptide sequence of the 19 kDa subunit of the luciferase produced by the shrimp Oplophorus gracilirostris.

5. The method as claimed in claim 4, wherein said exogenous enzyme is in a form with optimized activity and stability, sold by the company Promega under the name NanoLuc®.

6. The method as claimed in claim wherein the amount of exogenous enzyme released is measured by cleavage of a substrate specific for said enzyme, the substrate preferably being of the coelenterazine family, more particularly of the family of furimazine and derivatives thereof.

7. The method as claimed in claim 1, wherein the medium to be tested that surrounds the target cells comprises a biological agent and/or a chemical agent and/or a physical agent which is (are) potentially active with respect to said target cells.

8. The method as claimed in claim 7, wherein the biological agent is an antibody, preferably chosen from antibodies for therapeutic purposes, in particular monoclonal antibodies for therapeutic purposes.

9. The method as claimed in claim 8, wherein the antibodies are antibodies directed against molecules expressed mainly by normal or pathological B lymphocytes, such as CD19, CD20 or IL-6R (CD126), preferably CD20.

10. The method as claimed in claim 8, the antibodies are antibodies directed against molecules expressed mainly by normal or pathological T lymphocytes, such as CD3, CD25 or LPAM (Lymphocyte Peyer's Patch Adhesion Molecule or alpha[4]beta[7]-integrin).

11. The method as claimed in claim 8, wherein the antibodies are antibodies directed against molecules expressed mainly by normal or pathological cells of lymphoid origin, such as human CTLA-4 (CD152), PD-1 (CD279) or CD30.

12. The method as claimed in claim 8, wherein the antibodies are antibodies directed against molecules mainly expressed by normal or pathological cells of lymphoid and myeloid origin, such as human CD52, VLA4 (CD49d) or LFA-1 (CD11a).

13. The method as claimed in claim 8, wherein the antibodies are antibodies directed against molecules expressed mainly by carcinoid cells, such as EGFR (Epidermal Growth Factor Receptor, also denoted HER1 or ErbB1), EGFR2 (or HER2, or ErbB2), EGFR-3 (or HER3, or ErbB3) or EpCAM (CD326).

14. The method as claimed in claim 8, wherein the antibody is directed against the TNF-alpha molecule.

15. The method as claimed in claim 8, wherein the antibody is directed against the VEGF (Vascular Endothelial Growth Factor) molecule or its VEGFR receptor.

16. The method as claimed in claim 7, wherein the chemical and/or physical agent is chosen from chemotherapy agents or anti-cancer molecules, preferably cytotoxic molecules or molecules of the protein kinase inhibitor family.

17. The method as claimed in claim 1, wherein the target cells are genetically transformed in order to transiently or constitutively express said exogenous enzyme in a cytoplasmic and non-secreted form.

18. The method as claimed in claim 17, wherein the step of genetically transforming said target cells comprises introducing into said cells an expression vector carrying the coding sequence of the exogenous enzyme and a promoter of constitutive type which allows its transcription in a cell, such as a eukaryotic cell.

19. The method as claimed in claim 18, wherein the vector is a viral vector or a plasmid vector, preferably a plasmid vector.

20. The method as claimed in claim 18, wherein the exogenous enzyme expression vector comprises an antibiotic resistance gene.

21. The method as claimed in claim 20, wherein the antibiotic resistance gene is a gene for resistance to geneticin (G418), puromycin, blasticidin, hygromycin B, mycophenolic acid or zeocin, preferably a puromycin resistance gene.

22. The method as claimed in claim 19, wherein the introduction of said expression vector into said cells is carried out by infection with viral particles carrying the exogenous enzyme gene when the expression vector is a viral vector, or by chemical or physical methods when the expression vector is a plasmid vector.

23. The method as claimed in claim 22, wherein, when the vector is a plasmid vector, the introduction into the target cells is carried out by electroporation.

24. The method as claimed in claim 1, wherein said method is configured to measure the antibody-dependent cell-mediated cytotoxicity (ADCC), measuring the complement-dependent cytotoxicity (CDC) and/or measuring apoptosis.

25. A kit for carrying out the method as claimed in claim 1, wherein said kit in that it comprises:
a line of target cells expressing the exogenous enzyme,
cytotoxic effector cells for carrying out an ADCC test and/or a source of complement for carrying out a CDC assay,
a substrate which activates said exogenous enzyme so as to produce an emission of light, and any associated buffer solutions,
an instruction sheet.
